(19) United States
(12) Patent Application Publication
(10) Pub. No.: US 2022/0249441 A1
(43) Pub. Date: Aug. 11, 2022

(54) Homodimeric Tobramycin Adjuvant Repurposes Novobiocin as an Effective Antibacterial Agent Against Gram-Negative Bacteria

(71) Applicant: University of Manitoba, Winnipeg (CA)

(72) Inventors: Frank Schweizer, Winnipeg (CA); Temilolu Idowu, Winnipeg (CA)

(21) Appl. No.: 17/611,974

(22) PCT Filed: May 15, 2020
(86) PCT No.: PCT/CA2020/050656

§ 371 Date: Nov. 17, 2021

Related U.S. Application Data
(60) Provisional application No. 62/993,987, filed on Mar. 24, 2020.
(60) Provisional application No. 62/905,863, filed on Sep. 25, 2019.
(60) Provisional application No. 62/849,264, filed on May 17, 2019.

(51) Int. Cl. A61K 31/41; A61P 31/04; A61K 31/7036; A61K 31/7048; A61K 47/55

(52) U.S. Cl. CPC A61K 31/41; A61P 31/04; A61K 47/55; A61K 31/7048; A61K 31/7036

(57) ABSTRACT

Low permeability across the outer membrane is a major reason why most antibiotics are ineffective against Gram-negative bacteria. Agents that permeabilize the outer membrane are typically toxic at their effective concentrations. Here, we report the development of a broad-spectrum homodimeric tobramycin adjuvant that is non-toxic and more potent than the gold standard permeabilizing agent, polymyxin B nonapeptide. In pilot studies, the adjuvant confers potent bactericidal activity on novobiocin against Gram-negative bacteria, including carbapenem-resistant and colistin-resistant strains bearing plasmid-borne mcr-1 genes. Resistance development to the combination was significantly reduced, relative to novobiocin alone, and there was no induction of cross-resistance to other antibiotics, including the gyrase-acting fluoroquinolones. Tobramycin homodimer may allow the use of lower doses of novobiocin, overcoming its twin-problem of efficacy and toxicity.

### PRIOR APPLICATION INFORMATION

The instant application claims the benefit of U.S. Provisional Patent Application Ser. No. 62/849,264, filed May 17, 2019 and entitled “Homodimeric Tobramycin Adjuvant Repurposes Novobiocin as an Effective Antibacterial Agent against Gram-negative Bacteria”, the entire contents of which is incorporated herein by reference for all purposes.

The instant application also claims the benefit of U.S. Provisional Patent Application Ser. No. 62/905,863, filed Sep. 25, 2019 and entitled “Homodimeric Tobramycin Adjuvant Repurposes Novobiocin as an Effective Antibacterial Agent against Gram-negative Bacteria”, the entire contents of which is incorporated herein by reference for all purposes.

The instant application also claims the benefit of U.S. Provisional Patent Application Ser. No. 62/993,987, filed Mar. 24, 2020 and entitled “Homodimeric Tobramycin Adjuvant Repurposes Novobiocin as an Effective Antibacterial Agent against Gram-negative Bacteria”, the entire contents of which is incorporated herein by reference for all purposes.

### BACKGROUND OF THE INVENTION

The problem of antibacterial drug resistance is complex and multifaceted, and antibiotic drug development is being massively outpaced by emerging resistance against available antibiotics (1). Gram-negative bacteria are more difficult to eradicate because of their dual membrane topology and over expressed efflux pumps of broad substrate specificities (2, 3). The outer membrane (OM) of Gram-negative bacteria, which serves as a barrier to the permeation of potentially noxious molecules, including antibiotics, is composed of lipopolysaccharide (LPS) on the outer leaflet and phospholipid on the inner leaflet. The mechanism by which bacteria assemble this well-organized protective barrier has been well elucidated (4). To breach the OM, agents that interact directly with LPS stability (e.g. polybasic molecules such as polymyxins, aminoglycosides (AGs), and chelating agents such as EDTA) (5) or indirectly with mechanisms that synthesize, assemble, and/or transport the LPS (e.g. LpxC inhibitors, novobiocin, etc.) (6, 7) have been investigated as potential resistance breakers for Gram-negative bacteria.

Polybasic compounds destabilize the OM by displacing divalent metals that cross-bridge adjacent phosphate groups attached to LPS core sugar, thereby altering the well-ordered polyelectrolyte barrier. Truncation and/or modification of the polymyxin class of drugs has resulted into adjuvants such as polymyxin B nonapeptide (PMBN) (8) and SPR741 (9), where intrinsic antimicrobial activity was decoupled from their OM-destabilizing properties. Whereas PMBN seems to potentiate several OM-impermeable antibiotics against various colistin-susceptible Gram-negative bacteria (8), the activity of SPR741 excludes antipseudomonal effects (9). PMBN was shown to be generally less toxic than polymyxin B, but it causes similar proximal renal tubular injury in male rats (10). Similarly, conjugation and site-specific modification of AGs has resulted into scaffolds that lose the primary ribosomal properties of AGs but adopts an enhanced membrane effect (11-13). Among membrane-acting AGs, tobramycin-based adjuvants seem to be more effective against Pseudomonas aeruginosa than other Gram-negative bacteria (14). A major problem associated with the use of AGs is their propensity to cause irreversible hearing loss, an effect linked to the lack of precise selectivity for prokaryotic ribosomes (15-17). Synthetic AG analogs with lower bacterial and human mitochondrial ribosome specificities have been shown to exhibit reduced ototoxic potentials in cochlear explants, in culture and in guinea pig (18). Hence, non-ribosomal AGs may exhibit lower idiosyncratic toxicities and drug-induced hearing loss.

### SUMMARY OF THE INVENTION

According to an aspect of the invention, there is provided an antibiotic adjuvant compound comprising a structure as set forth in formula (I)

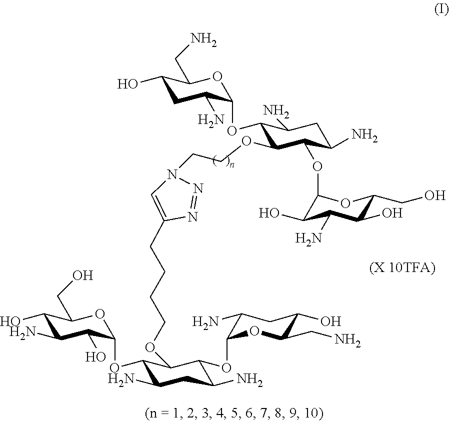

wherein n is an integer between 1 and 10.

According to an aspect of the invention, there is provided a method of permeabilizing a Gram Negative bacterium outer membrane comprising:

administering to the Gram Negative bacterium an effective amount of a compound comprising a structure as set forth in formula (I):

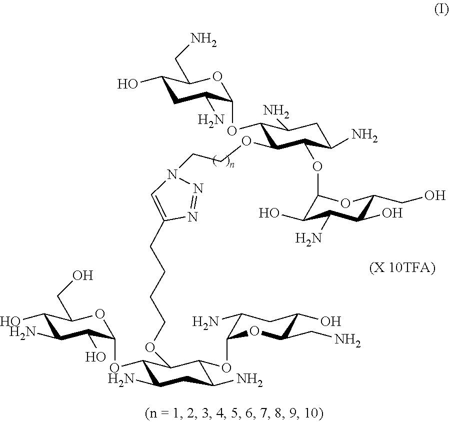

wherein n is an integer between 1 and 10.

According to an aspect of the invention, there is provided a method of potentiating antibacterial activity of an antibiotic against a Gram Negative bacterium comprising:

administering an effective amount of a compound of formula (I)

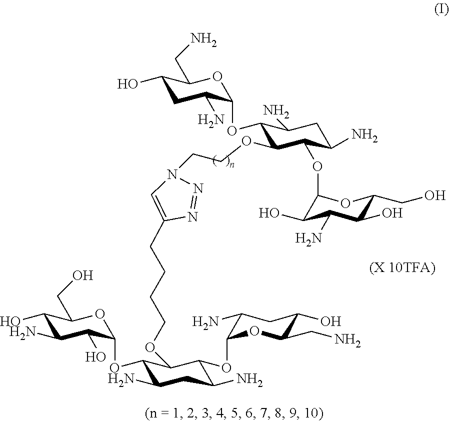

wherein n is an integer between 1 and 10; and an effective amount of an antibiotic selected from the group consisting of: an outer membrane impermeable antibiotic; an efflux-prone antibiotic; a β-lactam antibiotic; fosfomycin or a quorum sensing inhibitor such as a salicylanilide (e.g. niclosamide, rafoxanide, oxyclozanide or closantel).

The OM-impermeable antibiotic may be for example rifampicin, linezolid, clindamycin or novobiocin.

The efflux-prone antibiotic may be for example a tetracycline, a fluoroquinolone, chloramphenicol, or the like.

The β-lactam antibiotics may be for example a monobactam like aztreonam, a carbapenem like meropenem, a cephalosporin like ceftazidime or a penicillin like piperacillin.

In some embodiments there is provided the proviso that the antibiotic is not an aminoglycoside like tobramycin or a polymyxin like colistin.

### BRIEF DESCRIPTION OF THE DRAWINGS

FIG. 1. Structures of newly synthesized and reference compounds. Compounds 1-3 are tobramycin homodimers conjugated at the C-5 position of tobramycin with different tether lengths, compounds 4 and 5 are fragments of lead structure 1, and compound 6 is an aglycone derivative of novobiocin.

FIG. 2. Interactions of compounds 1-3 (at ≤7.1 μM) with different antibiotics against P. aeruginosa PAO1. FICI≤0.5=Green (synergistic); FICI>0.5 but<1=Yellow (no interaction); FICI>4=Red (antagonistic).

FIG. 3. Time-kill synergy graphs. The activities of novobiocin (32 μg/ml) in combination with compound 1 (16 μg/ml, i.e. 7.1 μM) against (A) P. aeruginosa PAO1, (B) K. pneumoniae 116381, (C) A. baumannii ATCC 17978, (D) E. coli ATCC 25922. Red bars and numbers indicate differences in bacterial concentrations between the starting inoculum and drug combination at 24 h. Purple bars and numbers indicate differences in bacterial concentrations between the combination and the most active single agent at 24 h (9 h for E. coli). The dashed lines represent the lower limit of detection. Each data point represents an average of three independent determinations.

FIG. 4. Outer membrane permeabilization by compound 1, polymyxin B nonapeptide (PMBN) and tobramycin (TOB) was determined by measuring the accumulation of 1-N-phenylnaphthylamine (NPN) in P. aeruginosa PAO1 cells. Each data point is an average of four independent determinations ±SD.

FIG. 5. Emergence of resistance study. Resistance acquisition during serial passaging of A. baumannii ATCC 17978 in the presence of sub-MIC levels of antibiotics. For combination study, compound 1 was kept constant at a concentration of 7.1 μM throughout the experiment.

FIG. 6. Preliminary efficacy studies of mono- and combination therapies in MDR Acinetobacter baumannii (AB)-challenged Galleria mellonella wax worms. A) AB LAC-4, a multidrug-resistant hypervirulent strain isolated from an hospital outbreak in LA county, California. B) AB 92247, a colistin-resistant MDR clinical isolate. Survivability of the larvae was scored every 6 h for 96 h.

FIG. 7. Scheme 1. Synthesis of tobramycin homodimers 1-3 and fragments 4-5. Conjugates differ in the length of tether.

FIG. 8. Scheme 2. Synthesis of novobiocin aglycone 6.

FIG. 9. Chemical structures of synthetic tobramycin- and nebramine-based adjuvants. TOB, tobramycin; NEB, nebramine; CIP, ciprofloxacin; g, guanidinylated. Blue represents tobramycin and nebramine fragments, green represents ciprofloxacin fragment and red represents cyclam fragment.

FIG. 10. Preparation of nebramine (NEB) homodimer 26. Reaction conditions: (a) reference [17], (b) i) 40% HCl in MeOH, 70° C., 48 h, ii) Boc₂O, Et₃N, MeOH/H₂O 2:1, v/v, 55° C., (c) TFA, DCM, rt, 1 h.

FIG. 11. Preparation of guanidinylated tobramycin-ciprofloxacin (gTOB-CIP) conjugate 7. Reaction conditions: (a) reference [19], (b) N,N′-diboc-N″-trifylguanidine, Et₃N, 1,4-dioxane/H₂O 3:1, v/v, rt, 70% (c) TFA, DCM, rt, 1 h, 90%

FIG. 12. Structure-activity relationship and lead optimization strategy of compound 1 (TOB-CIP).

FIG. 13. Triple combination studies between compounds 1-7 and (a) ceftazidime-avibactam, (b) aztreonam-avibactam, and (c) imipenem-relebactam. CAZ, ceftazidime; AVI, avibactam; IPM, imipenem; REL, relebactam. Dashed line represents CLSI susceptibility breakpoint.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

The problem of antimicrobial resistance is forcing a re-think and re-evaluation of our old antibiotics, with a view to repurposing them to combat emerging threats. Antibiotic adjuvants are becoming increasingly attractive as a strategy to address the immediate needs of effective treatment options against MDR pathogens. We report the development of a broad-spectrum homodimeric tobramycin adjuvant that is more potent than the gold standard potentiator molecule, PMBN, and confers potent bactericidal activity on novobiocin against MDR/XDR Gram-negative bacteria. A. baumannii isolates exhibit a high level of susceptibility to this combination, allowing the use of lower doses of novobiocin that could consequently improve tolerability and safety. Our data and others (26) suggest that intrinsic, but not acquired, resistance is the predominant mechanism of resistance to novobiocin in Gram-negative bacteria. Emergence of resistance to novobiocin alone by A. baumannii was spontaneous and conferred resistance to ciprofloxacin (consistent with target mutation) (41) whereas a combination of novobiocin and compound 1 suppressed this process and did not induce cross-resistance to other antibiotics. Preliminary in vivo evaluation in G. mellonella wax moths did not reveal any toxic interaction between the combination (colistin alone was toxic), but rather indicates a therapeutic potential for this combination.

Low permeability across the outer membrane is a major reason why most antibiotics are ineffective against Gram-negative bacteria. Agents that permeabilize the outer membrane are typically toxic at their effective concentrations. As discussed above, we have developed a homodimeric tobramycin adjuvant that is non-toxic and more potent than the gold standard permeabilizing agent, polymyxin B nonapeptide. As discussed herein, the adjuvant confers potent bactericidal activity on novobiocin against Gram-negative bacteria, including carbapenem-resistant and colistin-resistant strains bearing plasmid-borne mcr-1 genes. Resistance development to the combination was significantly reduced, relative to novobiocin alone, and there was no induction of cross-resistance to other antibiotics, including the gyrase-acting fluoroquinolones. Our tobramycin homodimer allows for the use of lower doses of novobiocin, overcoming its twin-problem of efficacy and toxicity.

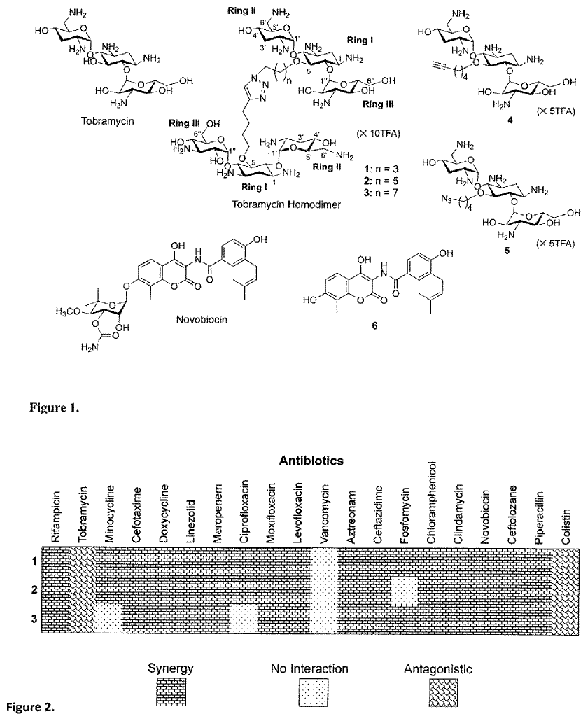

We sought to expand the spectrum of activity of tobramycin-derived adjuvants beyond P. aeruginosa by dimerizing the core scaffold of an amphiphilic tobramycin, such that the hydrophobic domain is sandwiched between two identical polar heads (FIG. 1). This was hypothesized to overcome the hemolytic problem of classic cationic amphiphiles (19-22). As discussed herein, we investigated the possibility of repurposing novobiocin against clinically-relevant Gram-negative bacteria using the newly synthesized adjuvant. Novobiocin (FIG. 1) is an orally active dihydroxy-glycosylated aminocoumarin antibiotic that inhibits DNA gyrase by binding the ATP-binding site in the ATPase subunit (7). In 2011, the oral form of novobiocin (novobiocin sodium capsule, 250 mg) was withdrawn from US market for “reasons of safety or effectiveness” (23). However, recent pharmacokinetic trials in non-infected subjects (Phase I and II studies) have demonstrated novobiocin plasma concentration of 150 μM (˜90-100 mg/L) for 24 h after a 5.5 g dose, with no serious toxicities (24, 25). Novobiocin displays limited activity against Gram-negative bacteria (MICs far higher than clinically achievable serum concentrations), even though their GyrB is sensitive to the antibiotic, due to the LPS-containing OM that act as a permeability barrier. PMBN had previously been investigated in combination with novobiocin to increase penetrance (26, 27), but effective concentrations and dose-limiting toxicities are of serious concern. Herein, we report the development of a non-toxic broad-spectrum antibiotic adjuvant that is more potent than PMBN and restores potent GyrB-dependent activity of novobiocin against multidrug (MDR) and extensively drug-resistant (XDR) Gram-negative bacteria. Concentrations as low as 0.25 μg/ml (0.1 μM) of the adjuvant were enough to cause a measurable effect and the addition of 7.1 μM resulted in the attainment of MICs levels below clinically achievable plasma concentration of novobiocin in all 28 isolates studied. We also provide insights into the mechanism of action and resistance development to this combination.

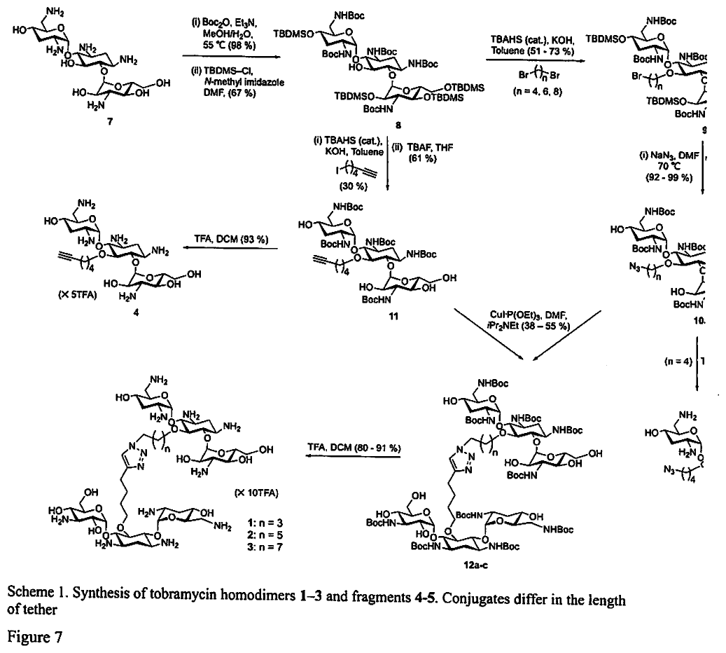

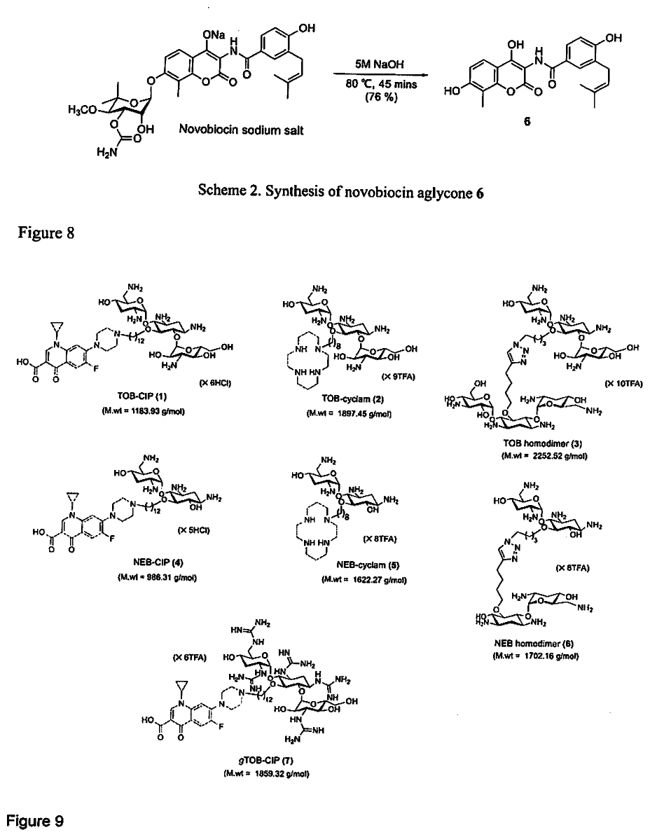

The design of tobramycin homodimers 1-3 was guided by previous SAR (13). Amphiphilic tobramycins with lipophilic groups at the 5-OH of deoxystreptamine (ring I; FIG. 1) have been shown to lose ribosomal activities but retain the ability to permeabilize the OM (13, 14). Dimerization of ribosome-targeting antibiotics has also been shown to result in poor inhibitors of in vitro protein translation (28). Hence, to prepare non-ribosomal amphiphilic-like tobramycin homodimers with potentially broad-spectrum OM permeabilizing properties, we dimerized two fragments of short-chain amphiphilic tobramycins ligated at the 4,6-disubstituted 2-deoxystreptamine via a copper(1)-catalyzed azide-alkyne cycloaddition reaction (FIG. 7, Scheme 1). This afforded regioselective 1,4-disubstituted 1,2,3-triazole products 1-3. Analogs with different tether length were synthesized to investigate the optimal spatial separation between the two domains while compounds 4 and 5 were prepared to study the SAR of the lead compound 1. The full synthetic strategy for preparing compounds 1-5 is outlined in Scheme 1 (FIG. 1). Compound 6 an aglycone derivative of novobiocin, was prepared to investigate the role of the L-noviose sugar on the gyrase activity of novobiocin (FIG. 8, Scheme 2).

β-Lactam antibiotics are currently the most used class of antibacterial agents in the infectious disease armamentarium [57]. However, the greatest challenge to this class of drugs is, arguably, the expression of hydrolysing bacterial enzymes that renders them ineffective and incapable of inhibiting PBPs. In P. aeruginosa, this problem is further exacerbated by the plethora of resources that the pathogen uses to subvert the potency of antibiotics. For instance, P. aeruginosa is capable of surviving harsh and low nutritional environments that may be inhospitable to other organisms [70], thus, reduced expression of OprD porin, low membrane permeability and overexpressed efflux pumps are important and common mechanisms of resistance in P. aeruginosa.

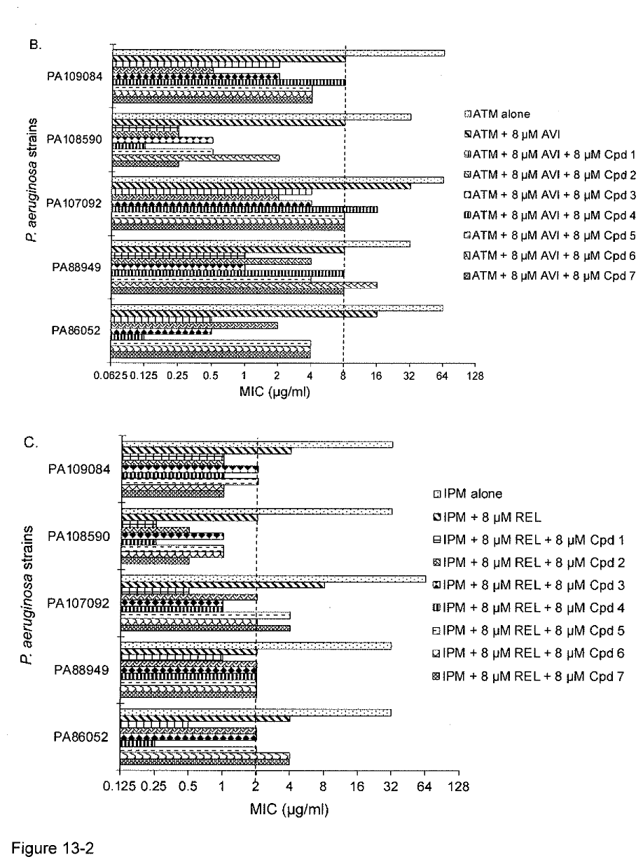

We developed an adjunctive combination strategy that rescued β-lactam antibiotics from multidimensional resistance in P. aeruginosa in vitro. Ligating a tobramycin scaffold to a secondary chemical entity (FIG. 9) resulted in the abrogation of intrinsic ribosomal properties of aminoglycosides, but an adoption of adjuvant capabilities. The C-5 position on the 4,6-disubstituted 2-deoxystreptamine ring of tobramycin was identified as optimal position for covalent ligation that preserves its adjuvant properties [13]. These tobramycin-based compounds, which are inactive by themselves, potentiate and restore potency to β-lactam antibiotics against carbapenem-resistant P. aeruginosa isolates when used in combination (Tables 7-9). None of these combinations had an antagonistic effect. It is noteworthy that the characteristic synergism between canonical aminoglycosides and β-lactams is typically due to the ribosomal properties of the former [48]. We also found that the inhibitory actions of β-lactamase inhibitors were further augmented by tobramycin-based adjuvants against P. aeruginosa isolates presumed to overexpress AmpC β-lactamase enzymes (FIG. 13). Hyperproduction of the intrinsic inducible chromosomal AmpC cephalosporinase is the main mechanism driving β-lactam resistance in P. aeruginosa [71]. Indeed, a triple combination of β-lactam plus β-lactamase inhibitor plus tobramycin-based adjuvants resulted in a 16- to 256-fold potentiation of the primary antibiotic (FIG. 13). Note that β-lactam/β-lactamase inhibitor combinations is the current state-of-the-art strategy utilized to preserve the therapeutic utility of β-lactam antibiotics [57], and microbiologic/clinical failures to these combinations are well documented [58,72,73].

We hypothesize the mechanism of β-lactam (+β-lactamase) potentiation is permeabilization of the OM of P. aeruginosa in a dose-dependent manner. This mechanism is consistent with the reversal of potentiation observed upon the addition of excess Mg²⁺ (MgCl₂) [48]. Moreover, it is unlikely that any of the secondary domain (e.g. ciprofloxacin, cyclam) co-ordinate with Zn²⁺ involved in MBL-dependent hydrolysis as the addition of excess Zn²⁺ (ZnSO₄) had no effect on potentiation. The ability to potentiate both monobactam (aztreonam) and carbapenems (imipenem, meropenem) also supports the hypothesis of an MBL-independent mechanism. Monobactams are resistant to MBLs while carbapenems are not [74].

We further assessed the role of efflux on these combinations and concluded that the involvement of RND efflux pumps is highly unlikely due to the ability of compounds 21-27 to potentiate β-lactam antibiotics in both wild-type and efflux-deficient mutants, almost to the same degree (Table 5). Unlike other β-lactam antibiotics, imipenem is not subject to efflux (consistent with our data in Table 9) and resistance in P. aeruginosa is often associated with the loss of OprD porin combined with activity of chromosomal AmpC β-lactamase [75,76]. In sum, the newly developed tobramycin-based conjugates potentiated β-lactam antibiotics to various extent against efflux-competent and efflux-deficient P. aeruginosa isolates while tobramycin by itself does not, suggesting that amphiphilicity and the overall physiochemical properties of the adjuvants are critical for the observed activity.

Described herein are a series of non-canonical tobramycin-based aminoglycoside conjugates that lose their ribosomal functions but adopt an adjuvant property. These molecules, when used in combination, restore the potency of β-lactam antibiotics against different recalcitrant P. aeruginosa phenotypes that exhibit multiple resistant mechanisms in vitro. We demonstrate that a triple combination of the newly synthesized adjuvant with a standard β-lactam/β-lactamase inhibitor combination offers better microbiological response than the current standard of care. Finally, we show that the potentiating effects of the newly reported molecules are unaffected by RND efflux pumps in P. aeruginosa, and that the effects are not replicated by monomeric units of tobramycin.

According to an aspect of the invention, there is provided an antibiotic adjuvant compound comprising a structure as set forth in formula (I)

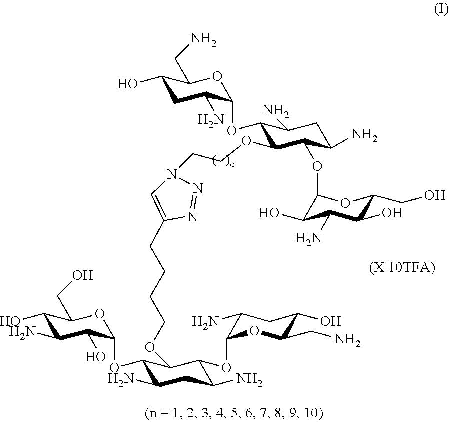

wherein n is an integer between 1 and 10.

According to an aspect of the invention, there is provided a method of permeabilizing a Gram Negative bacterium outer membrane comprising:

administering to the Gram Negative bacterium an effective amount of a compound comprising a structure as set forth in formula (I):

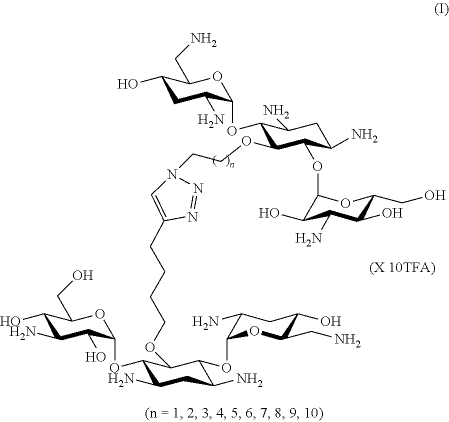

wherein n is an integer between 1 and 10.

According to an aspect of the invention, there is provided a method of potentiating antibacterial activity of an antibiotic against a Gram Negative bacterium comprising:

administering an effective amount of a compound of formula (I)

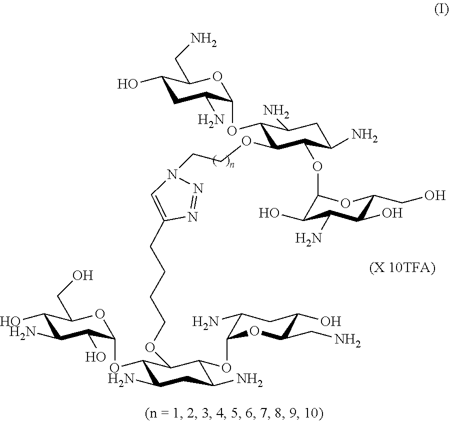

wherein n is an integer between 1 and 10; and an effective amount of an antibiotic selected from the group consisting of: an outer membrane impermeable antibiotic; an efflux-prone antibiotic; a β-lactam antibiotic; fosfomycin; or a quorum sensing inhibitor such as a salicylanilide (e.g. niclosamide, rafoxanide, oxyclozanide or closantel)

The OM-impermeable antibiotic may be for example rifampicin, linezolid, clindamycin or novobiocin.

The efflux-prone antibiotic may be for example a tetracycline, a fluoroquinolone, chloramphenicol, or the like.

The β-lactam antibiotic may be for example a monobactam like aztreonam, a carbapenem like meropenem, a cephalosporin like ceftazidime or a penicillin like piperacillin.

The β-lactam may be given or administered in combination with a β-lactamase inhibitor.

The β-lactamase inhibitor may be selected from the group consisting of: vaborbactam, sulbactam, clavulanic, tazobactam, zidebactam, nacubactam, ETX2514, avibactam and relebactam.

In some embodiments there is provided the proviso that the antibiotic is not an aminoglycoside like tobramycin or a polymyxin like colistin.

In some embodiments, the compound consists of or consists essentially of the chemical structure as set forth in formula (I). As will be appreciated by one of skill in the art, the compound consists essentially of this structure in that it may comprise chemical modifications thereto that do not affect or otherwise alter its adjuvant activity.

An effective amount is an amount of the adjuvant compound that is sufficient to increase outer membrane permeability of at least one Gram Negative bacterium. Outer membrane permeability can be determined using any of a variety of means known in the art, such as by determining changes in minimum inhibitory concentrations. Such an effective amount can be readily determined through routine experimentation.

As used herein, “permeabilizing”, “perturbing” and/or “disrupting” with reference to the outer membrane of a Gram-Negative bacteria all refer to methods for increasing the permeability of the outer membrane of a Gram-Negative bacterium compared to the outer membrane of a Gram-Negative bacterium of similar type that has not been modified or otherwise acted on, that is, a control Gram-Negative bacterium.

According to another aspect of the invention, there is provided a method of permeabilizing outer membranes of Gram-Negative bacteria comprising:

administering to the Gram Negative bacteria an effective amount of compound comprising a chemical structure of Formula (I), as set forth above.

As used herein, an “effective amount” of the compound is an amount of the compound that is sufficient to increase the permeability of the outer membrane of the Gram Negative bacteria compared to the outer membrane permeability of an untreated control as discussed above and as discussed in the examples below.

In some embodiments, the Gram Negative bacteria are within an individual. That is, in some embodiments of the invention, the method may comprise administering an effective amount of the compound to the individual.

As will be apparent to one of skill in the art, the individual has, is known to have, has been diagnosed as having or is suspected of having a bacterial infection caused by a Gram-Negative bacterium.

As discussed herein, the Gram-Negative bacterium is a multi-drug resistant Gram-Negative bacterium or an extensively-drug resistant Gram-Negative bacteria.

The multi-drug resistant Gram Negative bacteria may be selected from the group consisting of: MDR/XDR P. aeruginosa, MDR/XDR Acinetobacter baumannii, MDR/XDR Escherichia coli, MDR/XDR Klebsiella pneumoniae, and MDR/XDR Enterobacter cloacae.

The compound may be co-administered with an effective amount of an antibiotic. The antibiotic may be selected from the group consisting of: an outer membrane-impermeable antibiotic; an efflux-prone antibiotic; a β-lactam antibiotic; and Fosfomycin. Examples of outer-membrane impermeable antibiotics, efflux-prone antibiotics and β-lactam antibiotics are provided herein. Other examples will be readily apparent to one of skill in the art.

According to another aspect of the invention, there is provided use of a compound comprising a chemical structure of Formula (I), as set forth above for permeabilizing outer membranes of Gram-Negative bacteria.

The invention will now be further explained and/or elucidated by way of examples; however, the invention is not necessarily limited to or by the examples.

### Example 1—Chemical Synthesis of Tobramycin Homodimers (1-3), Fragments 4-5 and Novobiocin Aglycone (6)

The two amphiphilic tobramycin domains 4 and 5 were prepared following previously reported protocol (49). Tobramycin 7 was purchased from a commercial source and the amino groups were first protected using di-tert-butyl dicarbonate (Boc anhydride), followed by silylation of the N-Boc-tobramycin intermediate with excess TBDMSCI to afford a partially protected derivative 8 with free OH at the C-5 position of the deoxystreptamine ring. In the presence of a phase-transfer catalyst (TBAHS), 8 was alkylated with 1,n-dibromoalkane (n=4, 6, 8) in toluene to afford alkylated TBDMS-Boc-protected tobramycin intermediates 9a-c. Similarly, alkylation of 8 with iodohexyne under the same conditions followed by TBDMS deprotection afforded 11. The terminal bromine of 9a-c was then displaced by an azido nucleophile under anhydrous condition and the TBDMS protecting groups were deblocked using TBAF—to give compounds 10a-c, followed by Boc deprotection (using TFA) to give compound 5. Similarly, compound 4 was prepared by deblocking compound 11 with TFA. Dimerization of tobramycin was achieved by conjoining compounds 10a-c and 11 via “click chemistry” to afford 12a-c. Global deprotection of Boc-protecting groups using TFA afforded the final compounds 1-3 (FIG. 7, Scheme 1). Compound 6 was prepared by exposing novobiocin sodium salt to a highly basic condition (5M NaOH) at 80° C. (FIG. 8, Scheme 2).

### Example 2—Susceptibility and Toxicity Screening

Susceptibilities of different Gram-positive and Gram-negative bacteria to the newly synthesized molecules 1-3 were determined and compared to the progenitor molecule tobramycin. The lack of activity of compounds 1-3 (MIC≥16 vg/ml, Table S1) against a panel of organisms, relative to tobramycin, is consistent with loss of ribosomal binding. To investigate toxicity, we tested and found that compounds 1-3 were: i) non-hemolytic against porcine erythrocytes at 1024 vg/ml, ii) non-cytotoxic to human embryonic kidney (HEK293) and human liver carcinoma (HepG2) cells at 50 μM (>128 vg/ml), and iii) non-toxic in vivo against Galleria mellonella wax moths at 200 mg/kg. On the contrary, a single dose administration of 100 mg/kg colistin was toxic to G. mellonella and killed 90% of the larvae after 96 h.

### Example 3—Checkerboard Assay with Different Classes of Antibiotics

The lack of antibacterial activity and non-toxic properties of 1-3 further encouraged us to screen their adjuvant properties. An ideal adjuvant is a bioactive helper molecule that is inactive by itself but can potentiate the activity of a primary antibiotic and/or delay resistance development when used in combination. These types of molecules are less likely to select for resistance (29). To investigate this, checkerboard assay was used to assess the interactions between compounds 1-3 and nineteen different antibiotics (representing all major classes) against wild-type P. aeruginosa PAO1. P. aeruginosa was selected for this initial screen because OM permeability is a major mechanism of intrinsic resistance to antibiotics (30), and it is often regarded as a highly challenging model organism for new antibiotics (31). Compounds 1-3, investigated at ≤7.1 μM based on achievable plasma concentrations (20-200 μM) of aminoglycosides (32, 33), exhibit concentration-dependent synergistic relationships with all antibiotics tested against PAO1, except tobramycin, vancomycin, and colistin (FIG. 2). OM-impermeable antibiotics (such as rifampicin, linezolid, clindamycin and novobiocin), efflux-prone antibiotics (such as tetracyclines, fluoroquinolones, chloramphenicol, etc.), β-lactam antibiotics (such as monobactams, carbapenems, cephalosporins, and penicillins), and fosfomycin were all potentiated by 4- to 128-fold. Tobramycin by itself is not synergistic with these antibiotics. The antagonistic relationship between compounds 1-3 and tobramycin or colistin (FICI>4) is consistent with observed antagonism between tobramycin and colistin at high concentrations. This is perhaps due to competition for LPS binding by both polybasic molecules. The lack of potentiation of vancomycin is consistent with other OM permeabilizing agents such as PMBN and pentamidine (26, 34), where synergy is generally more pronounced with large hydrophobic molecules (e.g. rifampicin) than with large hydrophilic molecules (e.g. vancomycin). Compound 1 is the most potent and least toxic of the three, hence, it was used for further studies.

To investigate the spectrum of activity of the newly synthesized adjuvants, we examined a combination of compound 1 (at ≤7.1 μM) and novobiocin against MDR/XDR P. aeruginosa, Acinetobacter baumannii, Escherichia coli, Klebsiella pneumoniae, and Enterobacter cloacae exhibiting multiple resistance patterns. Novobiocin, an ATPase inhibitor of bacterial DNA gyrase and topoisomerase IV, was selected for this study because of its unique mechanism of action but lack of meaningful activity against Gram-negative bacteria even though their GyrB is sensitive to the antibiotic (26, 27). Synergy was retained in 100% of all isolates tested (8- to 256-fold potentiation; Table 1) including carbapenem- and colistin-resistant clinical isolates, isolates bearing GyrA mutations, and strains carrying the mobilized colistin resistance (mcr-1) gene that confer plasmid-mediated resistance to colistin (35). We conclude that resistance to novobiocin in Gram-negative bacteria is primarily due to OM permeability, and that resistance to fluoroquinolones (via GyrA mutations) does not necessarily confer resistance to novobiocin. To contextualize the potency of compound 1, we compared its ability to potentiate novobiocin to the ‘gold standard’ potentiator molecule, PMBN, and observed that at equimolar molar concentrations of 7.1 μM, compound 1 reproducibly displayed superior activity to PMBN against colistin-resistant Gram-negative bacteria (Table 2). Next, we investigated whether tobramycin by itself would potentiate novobiocin against Gram-negative bacteria. We tested a combination of tobramycin and novobiocin against tobramycin-susceptible and tobramycin-resistant Gram-negative bacteria strains and observed no synergy (FICI 0.75). Other antibiotics such as minocycline, ceftazidime, rifampicin, etc. also did not show any synergy with novobiocin, consistent with a previous chemical screen of 30,000 small molecules that identified just a handful of compounds (four hits) capable of synergizing with novobiocin (36). To establish whether the synergistic relationship of compound 1 with novobiocin was specific to Gram-negative bacteria, we assessed the combination against Gram-positive bacteria. Whereas novobiocin displayed potent activity against MRSA ATCC 33592 (MIC, 0.125 μg/ml), MRSE 61589 (MIC, 0.0625 μg/ml), and E. faecium ATCC 27270 (MIC, 2 μg/ml), consistent with prior literature (37), addition of compound 1 did not improve the activity of novobiocin further. This suggests that OM-permeabilization in Gram-negative bacteria, but not in Gram-positive, is the predominant mechanism by which compound 1 (and PMBN) enhances the activity of novobiocin. Notably, a high level of susceptibility to novobiocin (MIC≤1 μg/ml) was attained in wild-type and MDR A. baumannii clinical isolates in the presence of ≤7.1 μM of compound 1 (Table 1).

To investigate SAR of tobramycin homodimers, we prepared the constituent fragments (alkyne 4 and azide 5) of compound 1, the most potent of the synthesized derivatives (FIG. 7, Scheme 1). We also examined the adjuvant properties of a Boc-protected derivative 12a to investigate the role of free amines as it relates to OM destabilization. Compounds 4 and 5 did not potentiate novobiocin against P. aeruginosa PAO1, suggesting that covalent linkage of both domains is critical for the function of the adjuvant. This observation is consistent with optimal hydrophobic-charge threshold that must be maintained by cationic amphiphiles in order to destabilize bacterial membranes (19-22). Similarly, compound 12a did not potentiate novobiocin against P. aeruginosa PAO1, indicating that electrostatic interactions between the positively charged amines in compound 1 and the negatively charged phosphate residues on the OM of Gram-negative bacteria is central to the potency of the adjuvant.

### Example 4—Potentiation of Novobiocin is Independent of RND Efflux Pumps

To investigate whether efflux pumps play a role in the restoration of potent antibacterial activity of novobiocin by tobramycin homodimers, we assessed and compared the presence of synergy in efflux-deficient P. aeruginosa strains to wild type PAO1. Efflux mutants PAO200 and PAO750, lacking different clinically-relevant efflux pumps that extrude different classes of antimicrobial agents, exhibited higher level of susceptibilities to novobiocin (MIC, 32 μg/ml) than wild type PAO1 (MIC, 512 μg/ml), suggesting that novobiocin is a substrate of the RND efflux pumps. The MIC of compound 1 was unaffected by these pumps (Table 3). However, compound 1 (at 7.1 μM) further increased the susceptibilities of the mutants to novobiocin and lowered its MICs in both strains from 32 μg/ml to 0.125 μg/ml (256-fold potentiation) (Table 3). This suggests that whereas novobiocin is a substrate of the RND pumps, its potentiation by compound 1 is independent of these pumps.

### Example 5—Time-Kill Assay

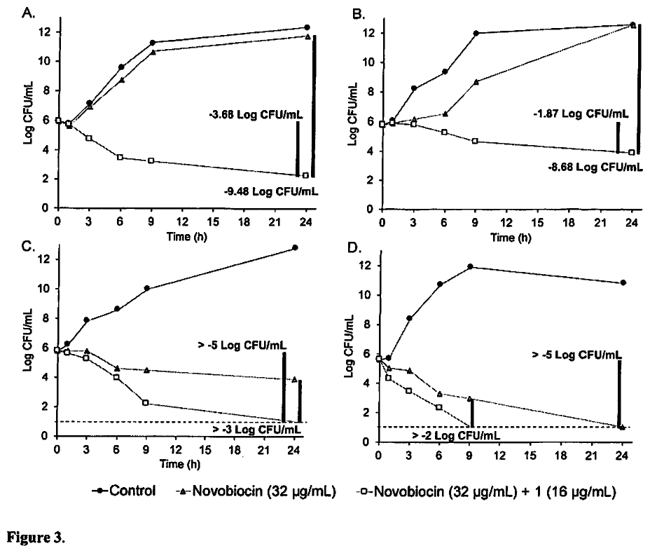

To scrutinize the data generated by checkerboard assay and provide more complementary evidence for the observed synergy, time-kill assays were performed on four different Gram-negative bacteria. Novobiocin is predominantly bacteriostatic versus most bacteria but could also be bactericidal against some pathogens. We investigated the kinetics of killing in LB media at fixed concentrations of 32 μg/ml (50.4μM) for novobiocin and 16 μg/ml (7.1 μM) for compound 1 because these concentrations (in combination) inhibit visible growth of all isolates studied in MHB media (Table 1). For PAO1 and K. pneumoniae 116381, 32 μg/ml of novobiocin alone resulted in bacterial growth identical to their respective controls (without drug) while a combination of novobiocin (32 μg/ml) and compound 1 (7.1 μM) inhibited the growth of both pathogens in LB media (FIG. 3). The combination resulted in bactericidal (PAO1) and synergistic relationships against both strains after 24 h. For A. baumannii ATCC 17978, 32 μg/ml novobiocin alone was bacteriostatic after 24 h, while a combination of novobiocin (32 μg/ml) and compound 1 (7.1 μM) was bactericidal (>3-Log reduction) and synergistic after 9 h of incubation (FIG. 3). After 24 h, the bacterial culture containing novobiocin and compound 1 was completely sterilized, representing >5-Log reduction from the starting inoculum. For E. coli ATCC 25299, 32 μg/ml novobiocin alone was bactericidal after 24 h, while a combination of novobiocin (32 μg/ml) and compound 1 (7.1 μM) exhibited bactericidal effects after 6 h of incubation (FIG. 3). Novobiocin and compound 1 became synergistic against E. coli ATCC 25922 after 9 h of incubation and the culture was completely sterilized within this period. Overall, the species-dependent degree of bacterial load reduction reflects, to a large extent, the fold potentiation of novobiocin by compound 1 in Checkerboard Assay.

### Example 6—Outer Membrane Permeabilization Assay

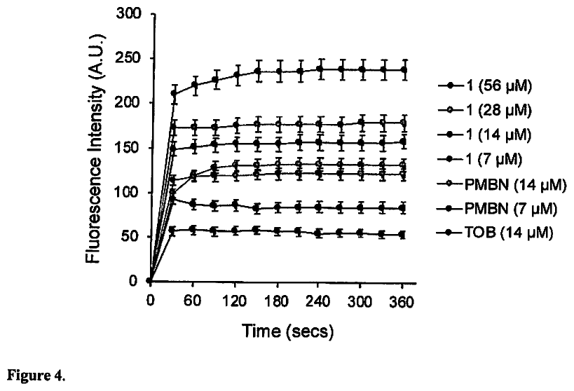

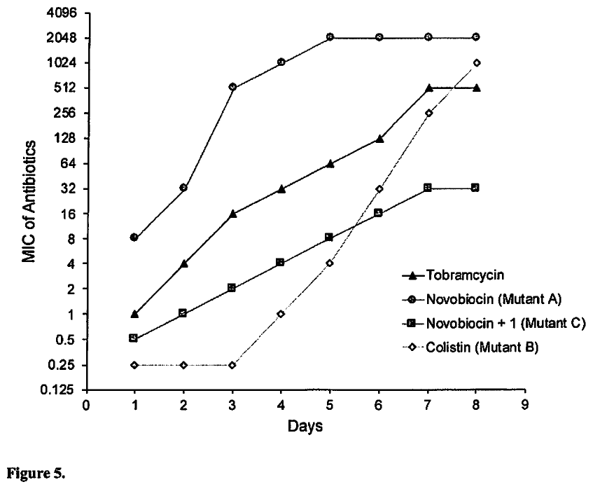

To investigate whether compound 1 enhances the uptake of novobiocin by permeabilizing the OM, we measured the intensity of fluorescence of the nonpolar probe 1-N-phenylnaphthylamine (NPN) using NPN uptake assay (40). An intact OM will ordinarily prevent the uptake of NPN dye which fluoresces strongly in phospholipid environments but only weakly in an aqueous environment. We observed that tobramycin homodimer 1 permeabilizes the OM of P. aeruginosa PAO1 in a dose-dependent manner, reminiscent of PMBN, while tobramycin alone showed weak fluorescence (FIG. 4). PMBN is a known OM permeabilizer (26), and our results showed that compound 1 (at 16 μg/ml) caused an increased fluorescence of NPN relative to PMBN (8 μg/ml) at similar micromolar concentrations (FIG. 4). This observation is consistent with data from checkerboard assay (Table 2).

### Example 7—Mechanism of Resistance Study

To investigate the possible mechanisms of resistance development to the combination, we generated some A. baumannii mutants by exposing wild-type ATCC 17978 to sub-MICs of novobiocin, alone and in combination with compound 1. A. baumannii was used for this study because all strains exhibited a high level of susceptibility to novobiocin+compound 1 combination (Table 1). Tobramycin-resistant and colistin-resistant mutants were also generated to investigate the mechanism and pattern of resistance development. Emergence of resistance to novobiocin alone was fast and high level, consistent with GyrB mutation (41), with a 64-fold change in MIC (from 8 μg/ml to 512 μg/ml) after just 3 days and a 256-fold change in MIC (from 8 μg/ml to 2048 μg/ml) after 5 days (FIG. 5). On the other hand, a combination of novobiocin and compound 1 (7.1 μM) resulted in a slow and steady 2-fold increase in MIC every generation, with a 4-fold increase in MIC (from 0.5 μg/ml to 2 μg/ml) after 3 days and a 16-fold change in MIC (from 0.5 to 8.0 μg/ml) after 5 days (FIG. 5). Tobramycin alone resulted in a 512-fold increase in MIC (from 1 to 512 μg/ml) after 7 days while colistin resulted in a 1024-fold change in MIC after 7 days (FIG. 5).

To gain insights into the emerging mechanism(s) of resistance to novobiocin/compound 1 combination, we assessed the susceptibilities of all generated mutants to different antibiotics (Table 4) and performed checkerboard assay on the novobiocin-resistant and colistin-resistant mutants A and B, respectively. Exposure of A. baumannii ATCC 17978 to sub-MIC levels of tobramycin homodimer 1 for seven serial passages did not generate tobramycin-resistant phenotype, whereas tobramycin alone did, suggesting that tobramycin homodimers do not bind to any critical target in the bacteria. Novobiocin-resistant mutant A (Table 4) was found to be resistant to ciprofloxacin (MIC, 16 μg/ml) and colistin (MIC, 8 μg/ml), suggesting gyrase mutation(s) as a primary mechanism of resistance but also LPS modification and efflux. Compound 1 was able to further potentiate novobiocin against this highly novobiocin-resistant mutant by 16-fold. Surprisingly, the generated colistin-resistant mutant B was hyper-susceptible to all antibiotics tested, consistent with a complete loss of LPS production that resulted in collateral sensitivity to antibiotics (42), or/and mutations in the PmrAB two-component system (43). The lipid A component of LPS is critical for the activity of colistin (42), and the development of such high level resistance to colistin under clinical conditions might constitute a huge fitness cost to the pathogen. PMBN and compound 1 did not potentiate novobiocin further against this strain, suggesting that LPS interaction is critical for OM destabilization by both compounds. Resistance to novobiocin/compound 1 combination did not confer resistance to tobramycin, ciprofloxacin, and colistin, but conferred low level resistance to novobiocin alone (Mutant C; Table 4), suggesting that the combination did not trigger the production of aminoglycoside modifying enzymes (AMEs), overexpression of efflux pumps and/or LPS modifications. By comparing the MICs of novobiocin versus mutants A and C, it is evident that compound 1 suppressed the development of resistance to novobiocin. Overall, the mechanism of resistance development to novobiocin/compound 1 combination is consistent with gyrase mutation, not LPS modification and AMEs production, and compound 1 significantly delayed this process.

### Example 8—Potentiation of Novobiocin by Compound 1 is Dependent on Gyrase B Activity

A study has shown that synergistic relationship between novobiocin and polymyxin B is independent of gyrase activity of novobiocin (44). To ascertain whether potentiation of novobiocin by compound 1 is indeed based on the binding of novobiocin to the ATP-binding site in the ATPase subunit of GyrB, we prepared novobiocin aglycone 6 (Scheme 2, FIG. 8) lacking the L-noviose sugar of novobiocin, which is known to make important contacts with GyrB (45). Consistent with a loss of gyrase activity, potent antibacterial activity was completely lost in compound 6, even against Gram-positive organisms that are susceptible to novobiocin. Novobiocin aglycone 6 has been reported to lose in vitro supercoiling activity against A. baumannii gyrase by at least 260-fold, relative to novobiocin (44). We thereafter evaluated whether any synergistic relationship exists between compounds 1 and 6 against P. aeruginosa PAO1, A. baumannii ATCC 17978, and E. coli ATCC 25922. Tobramycin homodimer 1 potentiates novobiocin against all of these strains (Table 1) but there was no potentiation of compound 6 against any of these pathogens (FICI>1). On the contrary, compound 6 was found to potentiate colistin against A. baumannii ATCC 17978 and E. coli ATCC 25922, similar to a previous report (44). This suggests that whereas the synergistic relationship between colistin and novobiocin (or compound 6) might not be gyrase-dependent, the synergistic relationship between tobramycin homodimers and novobiocin is DNA-gyrase dependent.

### Example 9—In Vivo Efficacy in Galleria mellonella Larvae

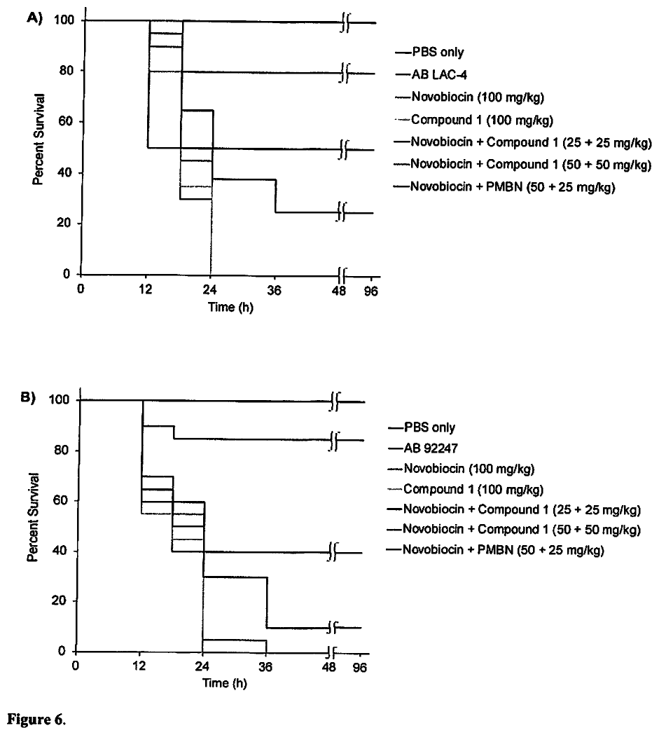

To expand the robustness of the data generated using in vitro assays, we performed preliminary in vivo investigation on the ability of novobiocin and compound 1 to protect G. mellonella moths from two different MDR A. baumannii infections. Despite its well-described limitations, the use of G. mellonella larvae as preliminary in vivo model system to study host-pathogen interactions, virulence, toxicity, and efficacy of novel compounds has been demonstrated with clinically used therapeutic agents (46-49). We found that the larvae tolerated 200 mg/kg each of compound 1 and novobiocin alone, and a 100+100 mg/kg combination of both, for more than 4 days, whereas exposure to 100 mg/kg of colistin (single dose administration) resulted in 90% mortality after 4 days. A multiple, but not single, dose administration of 25 mg/kg novobiocin+50 mg/kg PMBN had previously been reported to protect mice challenged with MDR P. aeruginosa or K. pneumoniae (26). In pilot studies, we investigated the in vivo therapeutic potential of novobiocin+compound 1 (25+25 and 50+50) mg/kg in the infection model. Untreated larvae challenged with MDR A. baumannii died within 24 h of infection. A single dose administration of 100 mg/kg each of novobiocin alone or compound 1 alone also resulted in 100% mortality of infected larvae after 24 h. However, a single dose administration of novobiocin+compound 1 (50+50 mg/kg) protected ˜80% of the larvae after 24 h for more than 96 h (FIG. 6). An approximate equimolar concentration of novobiocin+PMBN (50+25 mg/kg) only protected ˜35% of the larvae from the same infection (FIG. 6).

To ascertain that the combination therapy does not elevate toxicity against eukaryotic cells, we assessed the toxicity of a combination of compound 1 and novobiocin against HEK293 and HepG2 cell lines and found the combination to be non-cytotoxic.

### Example 10—Design and Chemistry

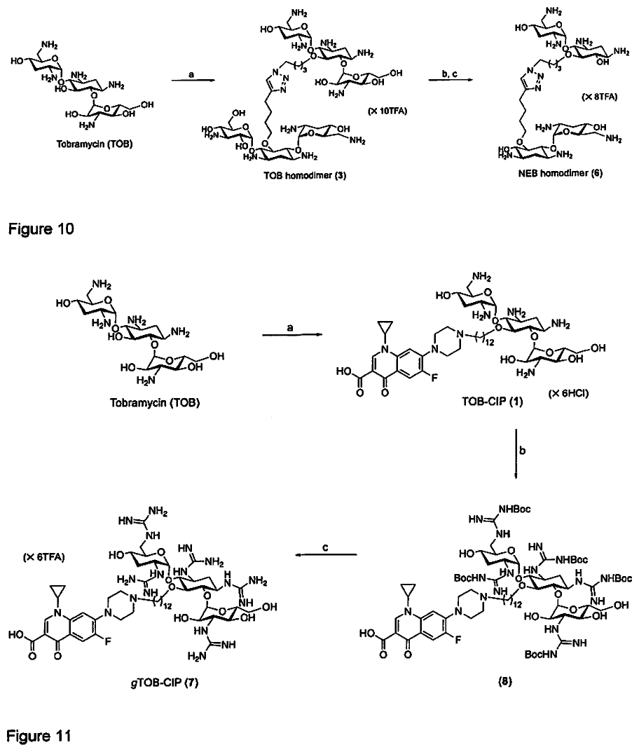

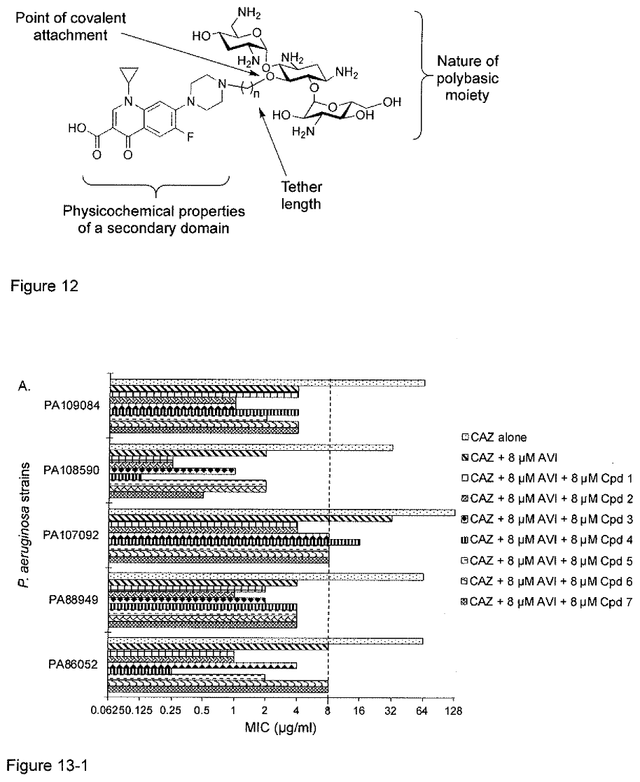

A series of tobramycin-based conjugates (21-23), corresponding nebramine-based conjugates (24-26), and guanidinylated tobramycin-ciprofloxacin conjugate 27 (FIG. 1) were prepared to abrogate the canonical ribosomal aminoglycoside activity but amplify the OM permeabilizing properties of amphiphilic aminoglycosides. In all conjugates, tobramycin was ligated to a secondary domain at the 4,6-disubstituted 2-deoxystreptamine ring via a hydrocarbon tether. Monomeric unit of tobramycin molecule served as a control for these molecules. Compounds 6 and 7 were prepared according to FIG. 10 and FIG. 11, respectively.

### Example 11 Structure-Activity Relationships and Lead Optimization

To further optimize compounds 21-23, corresponding nebramine-based derivatives 24-26 were prepared to reduce overall cationicity, such that OM-permeabilizing properties may be maintained while potential for in vivo toxicity may be diminished [59, 61]. In this study, we report the chemical synthesis and development of new derivatives 26 and 27 designed to further optimize compounds 23 and 21, respectively. Nebramine homodimer 26 was designed to reduce the number of positive charges and ribosomal target recognition of compound 23, with a view to eliminating aminoglycoside-related toxicity and resistance, while compound 27 was designed to introduce guanidine functional groups on compound 21. Guanidine is an important recurring amino acid in membrane-acting antimicrobial peptides and their introduction on the polyamines of tobramycin is an important chemical exploration to investigate the effect of increased basicity on antimicrobial activity. The synthetic strategies for preparing compounds 26 and 27 are outlined in FIGS. 10 and 11, respectively.

### Example 12 Antimicrobial Susceptibility Screening

The antimicrobial activities of compounds 21-27 and parent antibiotics (tobramycin and ciprofloxacin) were assessed against a panel of Gram-positive and Gram-negative bacteria. Whereas tobramycin and ciprofloxacin exhibited potencies against susceptible strains (as determined by CLSI susceptibility breakpoints), compounds 21-27 were mostly ineffective (MIC 16 μg/ml) against all isolates, indicating loss of intrinsic potency of parent molecules. For compounds 21-27, the loss of activity is important because bacterial resistance often arise as a consequence of selection pressure from antibiotic efficacy. Ciprofloxacin-ligated conjugates 21, 24 and 27 seem to exhibit some modest residual activity against some isolates, an effect believed to be mediated by the ciprofloxacin domain. Molecules prepared by ligation to fluoroquinolones at the N-piperazine ring often retain the activity of the respective fluoroquinolone domain [14,65]. Since compounds 21-27 were designed as adjuvants to overcome resistance in carbapenem-resistant P. aeruginosa, susceptibility of a wide range of MDR/XDR P. aeruginosa isolates to these molecules were also evaluated and were found to be non-susceptible (MICs 16 μg/ml). These MDR/XDR isolates exhibit multiple resistance patterns, with at least one strain (PA259-96918) expressing IMP-18 metallo-β-lactamase enzyme.

To investigate the role of efflux, we assessed the susceptibilities of efflux-deficient P. aeruginosa mutants (PAO200 and PAO750) to compounds 21-27 and compared with efflux-competent wildtype PAO1. PAO200 is a mexA-mexB-oprM deletion strain while PAO750 lacks five important RND pumps (MexAB-OprM, MexCD-OprJ, MexEF-OprN, Mex JK, and MexXY) and the outer membrane protein OpmH [38,39]. Some of these pumps are homologues of broad substrate specificities that extrude different classes of antimicrobial agents and confer resistance on P. aeruginosa [33]. Compounds 21, 24 and 27 displayed an MIC of 32 μg/ml each against wildtype PAO1 but an MIC of 4 μg/ml and 2 μg/ml each against PAO200 and PA0750, while the MICs of compounds 22, 23, 25 and 26 were identical in both wildtype and efflux-deficient mutants (Table 5). This suggests that ciprofloxacin-ligated compounds 21, 24 and 27 are substrates for RND efflux pumps while the lack of activity of compounds 22, 23, 25 and 26 is not due to the actions of RND efflux pumps. Functionalizing the tobramycin amino groups in compound 21 with guanidine in compound 27 seems to mitigate the effect of efflux pumps on TOB-CIP (Table 5).

### Example 13 Tobramycin/Nebramine-Based Conjugates Potentiate β-Lactam Antibiotics Against Carbapenem-Resistant P. Aeruginosa Isolates

The lack of intrinsic activity of compounds 21-27 prompted a further investigation of their ability to restore efficacy of β-lactam antibiotics against carbapenem-resistant Gram-negative bacteria. An ideal antibiotic adjuvant is a compound with no intrinsic activity in a syncretic combination [29]. Compounds 21-25 have been previously reported to potentiate OM-impermeable and efflux-susceptible antibiotics [48,59,60,61,13], but only compounds 22 and 25 have been investigated in combination with β-lactam antibiotics [48,59]. We hypothesized, based on our extensive studies with the MDR/XDR P. aeruginosa isolates under investigation, that a combination of β-lactamase enzymes, active efflux, low OM permeability and loss of porin channels are likely responsible for carbapenem-resistance in Gram-negative bacteria, especially in P. aeruginosa. Destabilization of the OM could therefore ensure that influx rate of β-lactam antibiotics overwhelms active extrusion and hydrolysis by β-lactamase, thereby enhancing bioaccumulation of intact molecules that may then bind to PBPs. Thus, we assessed the ability of compounds 21-27 to potentiate all subclasses of β-lactam antibiotics against wildtype P. aeruginosa PAO1, including monobactams (aztreonam), cephalosporins (ceftazidime), carbapenems (imipenem) and penicillins (piperacillin). We found that compounds 21-27 potentiate these β-lactam antibiotics to different degrees (2- to 64-fold) against this isolate (Table 6).

Next, we investigated whether compounds 21-27 can restore susceptibility of carbapenem-resistant P. aeruginosa isolates to aztreonam, ceftazidime and imipenem. We found that at a fixed concentration of 8 μM, compounds 21-23 potentiated all three antibiotics against all 13 carbapenem-resistant P. aeruginosa isolates tested, while compounds 24-27 had different combinatory interactions (synergistic and additive) with β-lactam antibiotics at similar concentrations (Tables 7-9). These interactions were generally dose-dependent. Note that a concentration of 8 μM is an easily achievable aminoglycoside concentration (20-200 μM) in human plasma [32,33]. For compound 21, susceptibilities equal to or below CLSI breakpoints were attained for aztreonam 8 μg/ml) in twelve out of thirteen resistant isolates, ceftazidime 8 μg/ml) in four out of eleven resistant isolates, and imipenem 2 μg/ml) in ten out of seventeen resistant isolates. For compound 22, CLSI breakpoints were reached for aztreonam in nine out of thirteen resistant isolates, ceftazidime in four out of eleven resistant isolates, and imipenem in four out of seventeen resistant isolates. For compound 23, CLSI breakpoints were attained for aztreonam in eleven out of thirteen resistant isolates, ceftazidime in four out of eleven isolates, and imipenem in five out of seventeen isolates. In contrast, the nebramine-derivatives 24-26 and guanidinylated compound 27 were less effective compared to their parent tobramycin-based molecules (Tables 7-9). This suggests that OM-permeabilization may overcome resistance to β-lactam antibiotics in P. aeruginosa, and that an amphiphilic tobramycin domain is better suited to achieve this than the pseudo-disaccharide nebramine and/or guanidinylated tobramycin. It is interesting to note that compounds 21 and 27, a guanidinylated derivative of each other, display similar MIC values but different OM-permeabilizing properties.

### Example 14 Efficacy of β-Lactam Antibiotics is Affected by Efflux, but Potentiation by Tobramycin/Nebramine-Based Conjugates is not

The effectiveness of β-lactam/β-lactamase inhibitor combinations may also be diminished by overexpressed efflux pumps, as much as reduced OM permeability. Efflux-deficient mutants PAO200 and PAO750 exhibit increased susceptibility to aztreonam and ceftazidime, but not imipenem (Tables 7-9), consistent with known contributions of MexAB-OprM pumps to intrinsic β-lactam resistance [67]. However, compounds 21-27 retained the ability to potentiate all β-lactam antibiotics tested against these mutants, almost to the same degree as in wildtype PAO1 (Tables 7-9), suggesting that the ability of compounds 21-27 to potentiate β-lactam antibiotics in P. aeruginosa is unaffected by the presence or absence of RND efflux pumps.

### Example 15—Triple Combination Studies with β-Lactam/β-Lactamase Inhibitors

P. aeruginosa often exhibit multiple resistance mechanisms to evade the actions of antibiotics [30]. To address this, we investigated the possibility of simultaneously permeabilizing the OM and inhibiting the actions of β-lactamase enzymes in a triple combination strategy. We also reasoned that since the rapidity with which β-lactamase inhibitors access their targets in the periplasm is critical for a successful inhibition, co-administration with OM permeabilizer may enhance their efficacy against MDR isolates. Thus, we investigated a combination of avibactam or relebactam ((3-lactamase inhibitors), compounds 21-27 (OM permeabilizers) and β-lactam antibiotics (aztreonam, ceftazidime or imipenem) against five P. aeruginosa isolates presumed to overexpress inducible AmpC (negative for the carbapenemases KPC, OXA-48, NDM, IMP, VIM and GES). All isolates were resistant to all three β-lactam antibiotics (Tables 7-9), while avibactam or relebactam (at ˜8 μM) potentiated the efficacy of these antibiotics by 2- to 16-fold (FIG. 13). Conversely, compounds 21-27 only marginally potentiated the tested β-lactam antibiotics against these isolates (Tables 7-9), suggesting a preponderance of enzymatic hydrolysis of β-lactam as opposed to reduced OM permeability. Remarkably, a combination of ˜8 μM each of avibactam and compounds 21-27 potentiated ceftazidime and aztreonam exponentially, and susceptibility far below CLSI susceptible breakpoints was attained in all isolates with at least one of such combinations (FIGS. 13a and 13b). Similarly, a combination of ˜8 μM each of relebactam and compounds 21-27 restored susceptibility to imipenem against these carbapenem-resistant isolates (FIG. 13c). Comparing potencies between compound 21-27, again we observed that tobramycin-based compounds appear to be generally more potent than the nebramine-based molecules.

### Example 16—Tobramycin Monomer does not Potentiate β-Lactam Antibiotics Against p. Aeruginosa Isolates at Clinically-Achievable Concentrations

There is a history of synergism between aminoglycosides and β-lactam antibiotics against P. aeruginosa isolates susceptible to one or both antibiotics, but not in isolates resistant to both [68,69]. We probed whether tobramycin by itself could potentiate the effects of β-lactam antibiotics against P. aeruginosa by investigating its interactions with aztreonam, ceftazidime and imipenem against wildtype, tobramycin-susceptible MDR, and tobramycin-resistant XDR phenotypes. Our results show that monomeric unit of tobramycin did not potentiate any of these antibiotics against wildtype (FICI=0.75-1.00) and tobramycin-susceptible MDR isolates (FICI=0.75). Against tobramycin-resistant MDR/XDR isolates, synergistic interactions were observed between imipenem and tobramycin at high concentrations (≥100 μM), but not between tobramycin and ceftazidime or aztreonam (FICI=0.56-0.75). At best, tobramycin only increased the susceptibility of tobramycin-resistant MDR/XDR strains to aztreonam and ceftazidime by 4- to 8-fold at one-half its MIC levels (up to 256 μg/ml). Note that high aminoglycoside concentrations are typically toxic and may be clinically unachievable in human plasma [32]. Overall, a very high concentration of tobramycin is needed, if at all, for a synergistic interaction with β-lactam antibiotics against MDR/XDR P. aeruginosa isolates.

### Experimental Section

Chemistry. All chemicals and reagents were purchased from Sigma-Aldrich (Oakville, ON, Canada) except tobramycin that was purchased from AK Scientific Inc. (CA, USA). The chemicals were all used without further purification. Air and moisture-sensitive reactions were performed with dry solvents under nitrogen atmosphere. Thin-layer chromatography (TLC) plates were visualized by staining within ninhydrin solution in n-butanol. Yields refer to chromatography-purified homogenous materials, except otherwise stated. ¹H and ¹³C NMR spectra were recorded on Bruker AMX-300 and AMX-500 spectrometers (Germany) as solutions and reported in the order of chemical shifts (δ) in ppm relative to the indicated solvent, multiplicity (s, singlet; d, doublet; t, triplet and m, multiplet), number of protons, and coupling constants (J) in hertz (Hz). ESI-MS and MALDI-TOF MS analyses were performed on Varian 500-MS ion trap mass spectrometer (USA) and Bruker Daltonics Ultraflextreme MALDI TOF/TOF mass spectrometer (Germany), respectively. Purity of final compounds, as determined by elemental analysis, was >95%.

General Procedure A: Global Amine Deprotection (Removal of Boc Protecting Groups) for Preparation of Compounds 1-3. Solution of Boc-protected compounds 12a-c in DCM (2.0 mL) were treated with trifluoroacetic acid (2.0 mL), stirred at RT for 1 h and concentrated under low vacuo. 2% methanol in diethylether (2.0 mL) was then added, stirred gently for 1 min and the solvent carefully decanted to give off-white solid compounds. The crude products were subsequently purified by reverse-phase flash chromatography (eluted with 100% deionized water) to afford analytically pure compounds 1-3 (80-91%) as off-white TFA salt solid compounds.

Tobramycin Homodimers 1-3. Final compounds 1-3 were prepared by global amine deprotection of compounds 12a-c, according to general procedure A. Tobramycin Homodimer 1. Yield (80%). ¹H NMR (500 MHz, D₂O) δ 7.75 (s, 1H, CH of triazole), 5.19 (d, J=2.5 Hz, 1H, anomeric H-1′), 5.17 (d, J=2.5 Hz, 1H, anomeric H-1′), 4.97 (d, J=2.7 Hz, 2H, anomeric H-1″), 4.30 (t, J=7.0 Hz, 2H, N—CH₂ of linker), 4.13-4.06 (m, 2H, H-5′), 3.96 (t, J=9.7 Hz, 2H, H-5″), 3.78-3.49 (m, 22H, H-5, H-6, H-4, H-2′, H-4′, H-2″, H-4″, H-6″, O—CH₂ of linker ×2), 3.44-3.31 (m, 6H, H-1, H-3, H-3″), 3.25-3.17 (m, 2H, H-6′), 3.14-3.08 (m, 2H, H-6′), 2.61 (m, 2H, CH₂ of linker), 2.36 (dt, J=12.9, 4.3 Hz, 2H, H-2), 2.09-2.00 (m, 4H, H-3′), 1.84-1.71 (m, 4H, H-2, CH₂ of linker), 1.57-1.41 (m, 6H, CH₂ of linker ×3), 1.19-1.16 (m, 2H). ¹³C NMR (125 MHz, D₂O) δ 163.28 (TFA), 163.00 (TFA), 162.72 (TFA), 162.44 (TFA), 124.16 (CH of triazole), 117.92 (TFA) 115.54 (TFA), 101.27 (C-1″), 101.17 (C-1″), 92.76 (C-1′), 82.09 (C-4″), 81.99 (C-4″), 81.80 (C-5), 81.77 (C-5), 77.07 (C-5″), 76.96 (C-5″), 76.14 (C-5′), 75.99 (C-5′), 73.21 (C-4), 73.18 (C-4), 73.06 (O—CH₂ of linker), 72.51 (O—CH₂ of linker), 68.55 (C-2″), 64.86 (C-6), 64.80 (C-6), 63.10 (C-4′), 59.35 (C-6″), 59.24 (C-6″), 54.82 (C-3″), 54.44, 50.86 (CH₂ of linker), 49.67 (C-1), 49.61 (C-1), 48.36 (C-3), 47.27 (C-2′), 47.24 (C-2′), 38.36 (C-6′), 38.32 (C-6′), 28.89 (CH₂ of linker), 27.98 (C-3′), 27.70 (C-2), 26.39 (CH₂ of linker), 25.99 (CH₂ of linker), 24.97 (CH₂ of linker), 24.12 (CH₂ of linker), 17.79, 16.32. MALDI: m/e calcd for C₄₆H₈₉N₁₃O₁₈H⁺, 1112.653; found 1112.650 [M+H]⁺.

Tobramycin Homodimer 2. Yield (85%). ¹H NMR (500 MHz, D₂O) δ 7.68 (s, 1H, CH of triazole), 5.21 (d, J=2.6 Hz, 1H, anomeric H-1′), 5.20 (d, J=2.6 Hz, 1H, anomeric H-1′), 4.99 (d, J=3.5 Hz, 2H, anomeric H-1″), 4.24 (t, J=7.1 Hz, 2H, N—CH₂ of linker), 4.14-4.07 (m, 2H, H-5′), 3.97 (td, J=9.8, 4.8 Hz, 2H, H-5″), 3.79-3.50 (m, 22H, H-5, H-6, H-4, H-2′, H-4′, H-2″, H-4″, H-6″, O—CH₂ of linker ×2), 3.46-3.34 (m, 6H, H-1, H-3, H-3″), 3.27-3.19 (m, 2H, H-6′), 3.18-3.08 (m, 2H, H-6′), 2.65-2.54 (m, 2H, CH₂ of linker), 2.37 (dt, J=12.8, 4.4 Hz, 2H, H-2), 2.14-1.97 (m, 4H, H-3′), 1.82-1.69 (m, 4H, H-2, CH₂ of linker), 1.60-1.41 (m, 6H, CH₂ of linker ×3), 1.21-1.11 (m, 4H, CH₂ of linker ×2). ¹³C NMR (125 MHz, D₂O) δ 163.37 (TFA), 163.09 (TFA), 162.81 (TFA), 162.53 (TFA), 123.62 (CH of triazole), 119.96 (TFA), 117.67 (TFA), 115.35 (TFA), 113.04 (TFA), 101.32 (C-1″), 101.30 (C-1″), 92.76 (C-1′), 81.96 (C-4″), 81.84 (C-5), 76.93 (C-5″), 76.88 (C-5″), 75.99 (C-5′), 75.89 (C-5′), 73.49 (O—CH₂ of linker), 73.18 (C-4), 73.13 (O—CH₂ of linker), 68.55 (C-2″), 64.79 (C-6), 64.78 (C-6), 63.14 (C-4′), 63.10 (C-4′), 59.23 (C-6″), 54.82 (C-3″), 50.59 (CH₂ of linker), 49.69 (C-1), 48.37 (C-3), 47.29 (C-2′), 47.25 (C-2′), 38.41 (C-6′), 38.34 (C-6′), 29.41 (CH₂ of linker), 29.28 (CH₂ of linker), 28.85 (CH₂ of linker), 28.05 (C-3′), 27.98 (C-3′), 27.71 (C-2), 25.83 (CH₂ of linker), 25.19 (CH₂ of linker), 24.65 (CH₂ of linker), 24.31 (CH₂ of linker). MALDI: m/e calcd for C₄₈H₉₃N₁₃O₁₈H⁺, 1140.684; found 1140.695 [M+H]⁺.

Tobramycin Homodimer 3. Yield (91%). ¹H NMR (500 MHz, D₂O) δ 7.73 (s, 1H, CH of triazole), 5.21 (d, J=2.6 Hz, 1H, anomeric H-1′), 5.20 (d, J=2.6 Hz, 1H, anomeric H-1′), 4.98 (d, J=3.4 Hz, 2H, anomeric H-1″)), 4.25 (t, J=7.0 Hz, 2H, N—CH₂ of linker), 4.10 (dt, J=8.4, 3.8 Hz, 2H, H-5′), 3.97 (td, J=9.8, 5.1 Hz, 2H, H-5″), 3.80-3.50 (m, 22H, H-5, H-6, H-4, H-2′, H-4′, H-2″, H-4″, H-6″, O—CH₂ of linker ×2), 3.46-3.33 (m, 6H, H-1, H-3, H-3″), 3.27-3.20 (m, 2H, H-6′), 3.16-3.09 (m, 2H, H-6′), 2.66-2.56 (m, 2H, CH₂ of linker), 2.37 (dt, J=12.5, 4.3 Hz, 2H, H-2), 2.09-2.01 (m, 4H, C-3′), 1.84-1.68 (m, 4H, H-2, CH₂ of linker), 1.61-1.41 (m, 6H, CH₂ of linker ×3), 1.19-1.06 (m, 8H, CH₂ of linker ×4). ¹³C NMR (125 MHz, D₂O) δ 163.35 (TFA), 163.07 (TFA), 162.79 (TFA), 162.50 (TFA), 123.92 (CH of triazole), 120.02 (TFA), 117.70 (TFA), 115.37 (TFA), 113.05 (TFA), 101.38 (C-1″), 101.27 (C-1″), 92.75 (C-1′), 81.97 (C-4″), 81.91 (C-5), 81.81 (C-4″), 76.90 (C-5″), 76.87 (C-5″), 75.96 (C-5′), 75.91 (C-5′), 73.75 (O—CH₂ of linker), 73.18 (C-4), 73.06 (O—CH₂ of linker), 68.56 (C-2″), 64.81 (C-6), 64.77 (C-6), 63.15 (C-4′), 63.12 (C-4′), 59.23 (C-6″), 59.21 (C-6″), 54.82 (C-3″), 50.98 (CH₂ of linker), 49.72 (C-1), 49.68 (C-1), 48.38 (C-3), 47.30 (C-2′), 47.25 (C-2′), 38.41 (C-6′), 38.35 (C-6′), 29.42 (CH₂ of linker), 29.30 (CH₂ of linker), 28.82 (CH₂ of linker), 28.78 (CH₂ of linker), 28.18 (CH₂ of linker), 28.06 (C-3′), 27.99 (C-3′), 27.70 (C-2), 25.57 (CH₂ of linker), 25.19 (CH₂ of linker), 25.09 (CH₂ of linker), 24.12 (CH₂ of linker). MALDI: m/e calcd for C₅₀H₉₇N₁₃O₁₈H⁺, 1168.715; found=1168.719 [M+H]⁺.

5-O-(Hexyne)-Tobramycin (4). Compound 4 was prepared by exposing compound 11 to TFA according to general procedure A. Yield (93%). ¹H NMR (500 MHz, D₂O) δ 5.40 (d, J=2.4 Hz, 1H, anomeric), 5.18 (d, J=3.4 Hz, 1H, anomeric), 4.26-4.22 (m, 1H), 4.07 (t, J=9.7 Hz, 1H), 3.97-3.71 (m, 11H), 3.61-3.51 (m, 2H), 3.48-3.44 (m, 1H), 3.44-3.29 (m, 3H), 2.49-2.45 (m, 1H), 2.45-2.40 (m, 1H), 2.33-2.25 (m, 3H), 2.25-2.16 (m, 1H, alkyne), 1.91 (m, 1H), 1.82-1.72 (m, 2H), 1.64-1.50 (m, 2H). ¹³C NMR (126 MHz, D₂O) δ 101.3 (anomeric), 92.7 (anomeric), 81.9, 81.8, 76.6, 75.6, 73.5, 73.2, 68.6, 64.9, 63.3, 59.3, 54.8, 51.2, 49.8, 48.5, 47.4, 38.6, 29.4, 28.2, 28.0, 27.8, 26.2, 25.3, 24.8, 19.4. MALDI TOF-MS m/e calcd for C₂₄H₄₅N₅O₉, 547.3217; measured me 548.3219 [M+H]⁺.

5-O-(4-Azidobutyl)-Tobramycin (5). Compound 5 was prepared by deprotecting compound 10a according to general procedure A. Yield (95%). ¹H NMR (300 MHz, D₂O) δ 5.39 (d, J=2.4 Hz, 1H, anomeric), 5.20 (d, J=3.4 Hz, 1H, anomeric), 4.34-4.17 (m, 2H), 4.00-3.71 (m, 11H), 3.63-3.52 (m, 3H), 3.49-3.27 (m, 4H), 2.61-2.48 (m, 1H), 2.36-2.19 (2H), 2.11-1.93 (m, 1H), 1.81-1.56 (m, 4H). ¹³C NMR (75 MHz, D₂O) 8101.1 (anomeric), 92.6 (anomeric), 81.9, 81.7, 76.6, 75.7, 73.1, 72.7, 68.5, 64.8, 63.2, 59.3, 54.8, 51.0, 49.6, 48.4, 47.3, 38.5, 28.0, 27.7, 26.7, 24.6. MALDI TOF-MS m/e calcd for C₂₂H₄₄N₈O₉, 565.3231; measured m/e 565.35541 [M+H]⁺.

Novobiocin Aglycone (6) was prepared as previously reported and NMR data were consistent with literature (44).

1,3,2′,6′,3″-penta-N-Boc-4′,2″,4″,6″-tetra-O-TBDMS-tobramycin (8). Commercial tobramycin 7 (4.00 g, 8.56 mmol) was dissolved in a 2:1 mixture of methanol and water (150 mL) and treated with Boc₂O (14.25 g, 65.29 mmol) in the presence of Et₃N (8.0 mL, 57.4 mmol). The reaction mixture was stirred under reflux (at 55° C.) overnight (˜20 h), concentrated under vacuo and thoroughly dried under high vacuum for 24 h to afford a white powdery solid (7.48 g, 90%). The dried crude penta-N-boc-protected tobramycin (1.04 g, 1.07 mmol) was dissolved in anhydrous DMF (6.0 mL) and treated with tert-butyldimethysilyl chloride, TBDMSCI (1.13 g, 7.49 mmol) and N-methylimidazole (0.6 mL, 7.49 mmol). The reaction was stirred at RT for 4 days under nitrogen gas atmosphere, and the resulting mixture was poured into water (50.0 mL) and extracted with DCM (50 mL, ×3). The organic layer was dried over anhydrous Na₂SO₄, concentrated in vacuo, and purified by flash chromatography using gradient elution (hexanes/ethyl acetate, 15:1 to 8:1, v/v) to afford 8 (1.05 g, 67%) as a white solid. NMR data are consistent with an earlier report (13).

### Compounds 21-25

Compounds 21-25 were synthesized as previously reported and NMR data were consistent with the earlier reports.

Nebramine Homodimer (26)

Tobramycin homodimer (23) (1 equiv.), was exposed to 40% HCl in MeOH at 70° C. for 48 h. The crude product was re-protected with Boc by dissolving in a 2:1 mixture of methanol and water and treated with Boc anhydride (20 equiv.), in the presence of triethylamine (20 equiv.). The reaction was stirred overnight (˜20 h) under reflux at 55° C. and concentrated under vacuo. The resulting mixture was then carefully purified by flash chromatography using gradient elution (hexanes/ethyl acetate, 1:1 v/v to 100% ethyl acetate). The resulting Boc-protected intermediate was subsequently treated with trifluoroacetic acid in DCM, stirred at rt for 1 h and concentrated under low vacuo. The crude product was crystallized in 2% methanol in diethyl ether as an off-white solid compound, and finally purified by reverse-phase flash chromatography (eluted with 100% deionized water) to afford analytically pure compound 6 as a white crystalline solid. ¹H NMR (500 MHz, D₂O) δ 7.75 (s, 1H, CH of triazole), 5.5 (d, J=2.5 Hz, 1H, anomeric H-1′), 4.40 (t, J=7.0 Hz, 2H, N—CF₂ of linker), 4.09-3.90 (m, 6H, H-5′), 3.85-3.49 (m, 13H, H-5, H-6, H-4, H-2′, H-4′, O—CH₂ of linker ×2), 3.44-3.25 (m, 6H, H-1, H-3), 2.78-2.69 (m, 2H, H-6′), 2.51 (m, 2H, CH₂ of linker), 2.25 (m, 2H), 2.10 (m, 2H), 2.00-1.80 (m, 6H, H-2 H-3′), 1.78-1.50 (m, 4H, H-2, CH₂ of linker). ¹³C NMR (125 MHz, D₂O) δ 163.40 (TFA), 163.12 (TFA), 162.83 (TFA), 162.55 (TFA), 123.67 (CH of triazole), 119.88 (TFA) 117.57 (TFA), 115.25 (TFA), 112.93 (TFA), 92.12 (C-1′), 82.76 (C-5), 82.73 (C-5), 75.25 (C-5′), 75.19 (C-5′), 73.46 (C-4), 73.35 (C-4), 72.87 (O—CH₂ of linker), 72.60 (O—CH₂ of linker), 72.58, 72.30, 63.64 (C-6), 50.33 (CH₂ of linker), 49.80 (C-1), 48.81 (C-3), 47.40 (C-2′), 39.07 (C-6′), 39.04 (C-6′), 28.85 (CH₂ of linker), 28.65 (C-3′), 27.85 (C-2), 26.34 (CH₂ of linker), 26.03 (CH₂ of linker), 25.01 (CH₂ of linker), 24.01 (CH₂ of linker). MALDI: m/e calcd for C₃₄H₆₇N₁₁O₁₀H⁺, 790.5072; found 790.5074 [M+H]⁺.

Guanidinylated Tobramycin-Ciprofloxacin Conjugate (27)

Tobramycin-ciprofloxacin (21) (1 equiv.), prepared as previously reported [13], was dissolved in a mixture of dioxane and water (3:1, v/v) and treated with di-Boc-trifylguanidine (15 equiv.) in the presence of triethylamine (15 equiv.). The reaction was stirred at rt for 7 days, concentrated in vacuo, extracted with DCM and dried over anhydrous Na₂SO₄. The organic layer was concentrated in vacuo and the crude product was purified by flash chromatography (dichloromethane/methanol, 80:1, v/v) to afford compound 28. Compound 28 was subsequently subjected to global amine deprotection (removal of Boc protecting groups) using TFA (in DCM), concentrated under low vacuo and crystallized using 2% methanol in diethyl ether. The crude product was finally purified by reverse-phase flash chromatography (eluted with 100% deionized water) to afford analytically pure compound 27 as a white amorphous solid. ¹H NMR (500 MHz, D₂O) δ 8.65 (s, 1H, N—CH of ciprofloxacin), 7.88 (d, J=12.9 Hz, 1H, C—CH of ciprofloxacin), 7.59 (d, J=7.5 Hz, 1H, C8-H of ciprofloxacin), 5.52 (d, J=2.5 Hz, 1H, anomeric H-1′), 5.30 (d, J=2.7 Hz, 1H, anomeric H-1″), 3.95-3.35 (m, 27H), 2.39-2.30 (m, 2H), 1.85-1.75 (m, 4H), 1.62-1.00 (m, 22H). ¹³C NMR (125 MHz, D₂O) δ 175.63 (CO of quinoline), 171.66 (CO of carboxylic acid), 163.40 (TFA), 163.12 (TFA), 162.84 (TFA), 162.56 (TFA), 158.55, 157.86, 156.61, 156.37, 155.95, 154.15 (CF of quinoline), 152.18 (CF of quinoline), 147.71 (CH, C-2 of quinoline), 143.04 (C-7 of quinoline), 138.73 (C-8a of quinoline), 119.86 (TFA), 117.54 (TFA), 115.22 (TFA), 112.90 (TFA), 111.78 (anomeric C-1″), 111.59 (anomeric C-1′), 106.67 (C-8 of quinoline), 99.98, 98.40, 94.73, 83.68, 78.72, 76.57, 74.49, 72.26, 71.93, 69.69, 67.53, 63.80 (C-4′), 59.82, 57.41 (N—CH₂ of linker), 56.77, 51.69, 51.26 (N—CH₂ of piperazine), 50.30 (C-1), 49.04 (C-3), 48.89 (C-2′), 46.99 (N—CH₂ of piperazine), 41.79 (C-6′), 35.16 (CH of cyclopropyl), 32.78, 32.44, 29.33 (O—CH₂—CH₂ of linker), 28.91 (CH₂ of linker), 28.78 (C-3′), 28.62 (CH₂ of linker), 28.49 (CH₂ of linker), 28.21 (CH₂ of linker), 27.97 (CH₂ of linker), 25.60 (CH₂ of linker), 25.00 (CH₂ of linker), 23.15 (CH₂ of linker), 7.55 (CH₂ of cyclopropyl). MALDI: m/e calcd for C₅₂H₈₇FN₁₈O₁₂Na⁺, 1198.1735; found 1198.1739 [M+Na]⁺.

General Procedure B: 5-O-Alkylation of Boc and TBDMS protected Tobramycin for the Preparation of Compounds 9a-c. A solution of 8 (1 equiv.) in toluene was treated with KOH (3 equiv.), 1,n-dibromoalkane (3 equiv.), and a catalytic amount of tetrabutylammonium hydrogen sulphate, TBAHS (0.1 equiv.). The reaction mixture was stirred at RT overnight, dispersed in water and extracted with an equal volume of ethyl acetate (×3). The organic layers were combined, washed with brine (×1), dried over anhydrous Na₂SO₄ and concentrated in vacuo. The crude products were then purified by column chromatography (hexanes/ethyl acetate, 12:1 to 10:1, v/v). to afford compounds 9a-c as white solids.

5-O-(n-Bromoalkyl)-1,3,2′,6′,3″-penta-N-Boc-4′,2″,4″,6″-tetra-O-TBDMS-tobramycin (9a-c). Compounds 9a, 9b, and 9c were prepared by treating 8 with 1,4-dibromobutane, 1,6-dibromohexane, and 1,8-dibromooctane, respectively, according to general procedure B.

5-O-(4-Bromobutyl)-1,3,2′,6′,3″-penta-N-Boc-4′,2″,4″,6″-tetra-O-TBDMS-tobramycin (9a). Yield (51%). ¹H NMR (300 MHz, CDCl₃) δ 5.24-5.12 (m, 2H, anomeric), 4.28-4.09 (m, 3H), 3.93-3.14 (m, 17H), 2.61-2.37 (m, 1H), 2.14-1.84 (m, 5H), 1.72-1.56 (m, 3H), 1.61-1.35 (m, 45H, Boc), 1.11-0.72 (m, 36H, TBDMS, tert-butyl), 0.24-0.09 (m, 24H, TBDMS —CH₃). ESI-MS: m/z calcd for C₇₁H₁₄₀BrN₅O₁₉Si₄Na+, 1583.2; found 1583.2 [M+Na]⁺.

5-O-(6-Bromohexyl)-1,3,2′,6′,3″-penta-N-Boc-4′,2″,4″,6″-tetra-O-TBDMS-tobramycin (9b). Yield (73%). ¹H NMR (300 MHz, Chloroform-d) δ 5.24-5.07 (m, 3H), 5.05-4.95 (m, 2H), 4.17-4.00 (m, 3H), 3.79-3.60 (m, 5H), 3.60-3.26 (m, 11H), 3.24-3.15 (m, 2H), 2.47-2.32 (m, 1H), 1.98-1.88 (m, 1H), 1.82-1.69 (m, 3H), 1.54-1.24 (m, 52H), 0.96-0.73 (m, 36H), 0.15--0.10 (m, 24H). ¹³C NMR (75 MHz, CDCl₃) δ 96.34, 85.70, 79.81, 79.29, 79.16, 79.11, 75.20, 73.04, 71.49, 68.03, 66.96, 63.09, 57.18, 50.50, 48.85, 48.27, 36.66, 35.67, 33.54, 33.32, 32.73, 30.32, 28.57, 28.43, 28.35, 27.19, 26.06, 25.93, 25.73, 25.23, 24.67, 18.40, 18.23, 18.02, 17.84, −3.54, −3.85, −4.26, −4.94, −4.98, −5.10, −5.21, −5.26. MALDI: Exact mass calcd for C73H144BrN5O19 Si4Na⁺, 1608.861; found 1608. 886[M+Na]⁺.

5-O-(8-Bromooctyl)-1,3,2′,6′,3″-penta-N-Boc-4′,2″,4″,6″-tetra-OTBDMS-tobramycin (9c). Yield (60%). ¹H NMR (300 MHz, CDCl₃) δ 5.26-5.12 (m, 2H, anomeric), 4.39-3.97 (m, 3H), 3.89-3.07 (m, 16H), 2.47 (d, J=12.8 Hz, 1H), 2.08-1.94 (m, 2H), 1.93-1.77 (m, 2H), 1.65 (m, 1H), 1.56-1.39 (m, 45H, Boc), 1.38-1.14 (m, 8H), 1.13 (m, 1H), 1.05-0.75 (m, 36H, TBDMS tert-butyl), 0.34-0.15 (m, 24H, TBDMS—CH₃). ¹³C NMR (75 MHz, CDCl₃) δ 85.77, 79.42, 79.24, 57.27, 50.52, 34.06, 32.88, 32.83, 31.60, 30.67, 30.05, 29.67, 29.58, 29.47, 29.44, 28.82, 28.66, 28.52, 28.42, 28.22, 28.18, 26.16, 26.04, 26.01, 25.80, 18.52, 18.36, 18.12, 17.93, −3.38, −3.77, −4.17, −4.93, −5.06, −5.21. ESI-MS: m/z calcd for C₇₅H₁₄BrN₅O₁₉Si₄Na⁺, 1636.89; found 1636.80 [M+Na]⁺.

General Procedure C: Conversion of Bromoalkyl to Azide. A solution of bromoakylated compounds 9a-c (1 equiv.) in anhydrous DMF was treated with sodium azide (10 equiv.) and stirred at 70° C. for 3 h. The resulting mixture was concentrated in vacuo, re-dispersed in water and extracted with ethyl acetate. The combined organic layers were subsequently washed with brine (×1), dried over anhydrous Na₂SO₄ and concentrated in vacuo to give yellow solids.

General Procedure D: Deprotection of Hydroxyl Groups (Removal of TBDMS Protecting Groups). A solution of TBDMS- and Boc-protected compounds in anhydrous THF (5.0 mL) were treated with tetrabutylammonium fluoride (TBAF, 6 equiv.) and stirred under nitrogen atmosphere for 2 h. The reaction mixture was concentrated under vacuo, dissolved in water and extracted with DCM (×3). The organic layers were combined, dried over anhydrous Na₂SO₄, concentrated in vacuo, and purified by column chromatography (hexanes/ethyl acetate, 1:1, v/v, then dichloromethane/methanol, 25:1 to 20:1, v/v) to afford off-white solids.

5-O-(n-Azidoalkyl)-1,3,2′,6′,3″-penta-N-Boc-tobramycin (10a-c). Compounds 10a-c were prepared by converting 9a-c to azido compounds, according to general procedure C, and subsequently removing the TBDMS protecting groups according to general procedure D.

5-O-(4-Azidobutyl)-1,3,2′,6′,3″-penta-N-Boc-tobramycin (10a). Overall yield (45%). ¹H NMR (300 MHz, CDCl₃) δ 5.35-5.27 (m, 1H), 5.19-5.09 (m, 1H), 3.98-3.59 (m, 12H), 3.55-3.35 (m, 4H), 3.35-3.19 (m, 3H), 2.24-2.05 (m, 2H), 1.73-1.58 (m, 5H), 1.55-1.33 (m, 45H), 1.31-1.12 (m, 4H). ESI-MS: m/z calcd for C47H84N8O19Na⁺, 1087.58; found 1087.61 [M+Na]⁺.

5-O-(6-Azidohexyl)-1,3,2′,6′,3″-penta-N-Boc-tobramycin (10b). Overall yield (65%). ¹H NMR (300 MHz, CDCl₃) δ 5.33-5.12 (m, 3H), 4.31-4.09 (m, 1H), 3.96-3.53 (m, 12H), 3.48-3.33 (m, 3H), 3.31-3.05 (m, 4H), 2.22-2.01 (m, 2H), 1.76-1.46 (m, 6H), 1.46-1.08 (m, 52H). ¹³C NMR (75 MHz, CDCl₃) δ 80.59, 80.01, 79.40, 78.06, 73.25, 72.42, 70.23, 64.99, 62.08, 56.52, 52.33, 51.31, 49.10, 33.12, 29.66, 28.67, 28.47, 28.40, 28.33, 26.68, 25.21, 20.07, 13.53. ESI-MS: m/z calcd for C₄₉H₈₈N₈O₁₉Na⁺,1116.3; found 1116.9 [M+Na]⁺.

5-O-(8-Azidooctyl)-1,3,2′,6′,3″-penta-N-Boc-tobramycin (10c). Overall yield (57%). ¹H NMR (300 MHz, CDCl₃) δ 5.49-5.35 (m, 1H), 5.25-5.11 (m, 1H), 3.88-3.30 (m, 16H), 3.23-3.02 (m, 4H), 2.14-1.90 (m, 2H), 1.73-1.04 (m, 62H). ¹³C NMR (75 MHz, CDCl₃) δ 158.77, 157.88, 155.70, 155.30, 155.09, 96.60, 80.36, 79.83, 79.50, 79.20, 73.21, 70.31, 64.94, 61.95, 56.33, 53.42, 51.30, 48.99, 32.98, 29.76, 29.53, 29.38, 28.94, 28.68, 28.44, 28.34, 28.27, 28.03, 26.49, 25.53. MALDI: m/e calcd for C₅₁H₉₂NaO₁₉Na⁺, 1143.638; found 1143.659 [M+Na]⁺.

5-O-Hexyne-1,3,2′,6′,3″-penta-N-Boc-tobramycin (11). Compound 11 was prepared by reacting 8 with 6-iodohexyne, according to general procedure B, followed by TBDMS deprotection following general procedure D. Yield (30%). ¹H NMR (500 MHz, CDCl₃) δ 5.31-5.23 (m, 2H), 5.15-4.97 (m, 2H), 3.95-3.50 (m, 14H), 3.48-3.34 (m, 4H), 3.29-3.20 (m, 1H), 3.18-3.06 (m, 1H), 2.24-2.06 (m, 4H), 1.97-1.93 (m, 1H, alkyne), 1.73-1.62 (m, 3H), 1.60-1.31 (m, 49H), 1.27-1.20 (m, 2H). MALDI: m/e calcd for C₄₉H₈₅N₅O₁₉Na⁺, 1070.574; found 1070.596 [M+Na]⁺.

General Procedure E: Copper (1)-catalyzed azide-alkyne cycloaddition reaction (“Click Chemistry”) for the Preparation of compounds 12a-c. Compounds 10a-c (2 equiv.) and 11 (1 equiv.) were dissolved in an anhydrous DMF (4.0 mL) and treated with CuI.P(OEt)₃ (3 equiv.) and iPr₂NEt (3 equiv.) The reaction was stirred under nitrogen gas for 2 h. The reaction mixture was concentrated under vacuo, dissolved in water and extracted with DCM (×3). The organic layers were combined, dried over anhydrous Na₂SO₄, concentrated in vacuo, and purified by column chromatography (dichloromethane/methanol, 40:1 to 20:1, v/v) to afford compounds 12a-c (38-55%) as white solids.

5-O-Alkylated-1,3,2′,6′,3″-penta-N-Boc-tobramycin homodimers (12a-c). Compounds 12a-c were prepared via a copper(1)-catalyzed azide-alkyne cycloaddition reaction between 10a-c and 11, according to general procedure E.

Compound 12a. Yield (38%). ¹H NMR (300 MHz, CDCl₃) δ 5.39-4.99 (m, 4H), 4.32 (s, 2H), 3.88-3.33 (m, 27H), 3.15 (m, 2H), 2.87-2.52 (m, 2H), 2.45-2.02 (m, 5H), 2.03-1.81 (m, 1H), 1.66-1.05 (m, 103H). MALDI: m/e calcd for C₉₆H₁₉N₁₃O₃₈Na⁺, 2135.159; found 2135.169 [M+Na]⁺.

Compound 12b. Yield (55%). ¹H NMR (300 MHz, CDCl₃) δ 5.38-4.98 (m, 4H), 4.42-4.21 (m, 1H), 4.02-3.24 (m, 25H), 3.23-2.88 (m, 2H), 2.78-2.56 (m, 1H), 2.32-2.07 (m, 2H), 1.98-1.82 (m, 2H), 1.69-0.88 (m, 106H). MALDI: m/e calcd for C₉₈H₁₇₃N₁₃O₃₈Na⁺, 2164.514; found 2164.519 [M+Na]⁺.

Compound 12c. Yield (42%). ¹H NMR (500 MHz, CDCl₃) δ 7.37 (s, 1H), 5.28-5.11 (m, 5H), 4.28 (t, J=6.5 Hz, 2H), 4.13 (s, 2H), 3.93-3.25 (m, 34H), 3.20-3.00 (m, 2H), 2.80-2.63 (m, 2H), 2.48 (s, 3H), 2.29-1.98 (m, 4H), 1.93-1.77 (m, 2H), 1.71-1.04 (m, 108H). ¹³C NMR (125 MHz, CDCl₃) δ 158.83, 157.95, 155.99, 155.24, 155.17, 121.36, 96.64, 80.49, 80.04, 79.42, 79.38, 73.16, 70.88, 70.58, 65.21, 56.47, 53.39, 50.59, 50.09, 49.26, 49.15, 40.88, 33.20, 31.89, 29.66, 29.19, 28.47, 28.44, 28.42, 28.39, 28.34, 25.16, 24.97, 22.65, 14.08. MALDI: m/e calcd for C₁₀₀H₁₇₇N₁₃O₃₈Na⁺, 2191.222; found 2191.233 [M+Na]⁺.

Microbiology. Bacteria isolates were either obtained from the American Type Culture Collection (ATCC), the Canadian National Intensive Care Unit (CAN-ICU) surveillance study (50), or the Canadian Ward (CANWARD) surveillance study (51, 52). Clinical isolates obtained as part of the CAN-ICU and CANWARD studies from participating medical centers across Canada were cultured from body fluids and tissues of patients suffering from presumed “clinically significant” infectious diseases. Antimicrobial susceptibilities of clinical isolates were evaluated (using ATCC strains as quality control strains) and categorized, where appropriate, as either multidrug resistant (MDR), extensively drug-resistant (XDR), or pan drug-resistant (PDR). MDR is defined as acquired non-susceptibility to at least one agent in three or more antimicrobial categories, XDR as non-susceptibility to at least one agent in all but two or fewer antimicrobial categories (i.e. bacterial isolates remain susceptible to only one or two categories), and PDR as non-susceptibility to all agents in all antimicrobial categories (53).

Antimicrobial Susceptibility Assay. The in vitro antimicrobial activity of all compounds/antibiotics against a panel of bacteria was evaluated by microbroth dilution method in accordance with the Clinical and Laboratory Standards Institute (CLSI) guidelines (using ATCC strains as quality control strains) (54). Overnight grown bacterial cultures were diluted in saline to achieve a 0.5 McFarland turbidity, followed by 1:50 dilution in Mueller-Hinton broth (MHB) for inoculation to a final concentration of approximately 5×10⁵ CFU/mL. The antimicrobial agents were 2-fold serially diluted in MHB in a 96-well plate and incubated at 37° C. with equal volumes of inoculum for 18 h. The lowest concentration that prevented the visible growth of bacteria was defined as the MIC for each antimicrobial agent. The broth with or without bacterial cells was used as positive or negative control, respectively.

Checkerboard Assay. Combination studies with different antibiotics were performed in 96-well plates as previously described (19). Briefly, the antibiotic of interest was serially diluted in MHB along the abscissa while the adjuvant (tobramycin homodimer) was serially diluted in MHB along the ordinate. This creates a 10×7 matrices wherein each well consists of a combination of different antibiotic and adjuvant concentrations. Overnight grown bacterial cultures were diluted in saline to achieve a 0.5 McFarland turbidity, followed by 1:50 dilution in Mueller-Hinton broth (MHB) for inoculation to a final concentration of approximately 5×10⁵ CFU/mL. Equal volume of this bacterial culture was then added to each well and incubated at 37° C. for 18 h. After incubation, the plates were read on EMax® Plus microplate reader (Molecular Devices, Sunnyvale, Calif., USA) at 590 nm. MIC was recorded as wells with the lowest concentration of drugs with no bacterial growth. The fractional inhibitory concentration (FIC) for each antibiotic was calculated by dividing the MIC of the antibiotic in the presence of adjuvant by the MIC of the antibiotic alone. Similarly, the FIC of adjuvant was calculated by dividing the MIC of the adjuvant in the presence of antibiotic by the MIC of the adjuvant alone. FIC index is the sum of both FICs. FIC indices of ≤0.5 were deemed synergistic; >0.5-4, no interaction; and >4, antagonistic.

Cytotoxicity Assay. Human embryonic kidney cells (HEK293) and HepG2 cells were grown in Dulbecco's modified eagle's medium supplemented with 10% fetal bovine serum in a humidified 5% atmospheric incubator at 37° C. Equal number of cells (100 μl) of media containing ˜8000 cells) were dispersed into 96-well plates and wells with medium but no cells were used as blanks. After incubating for 24 h, 100 μl of varying concentrations of test compounds (at twice the desired concentrations) were added to each well, including the blanks. The treated cells were then incubated further for 48 h, after which PrestoBlue reagent was added to each well. The plates were then incubated for an additional hour on a nutator mixer in a 5% C02 incubator. The fluorescence was read at 490 nm on a SpectraMax M2 plate reader (Molecular Devices, USA). Cell viability were interpreted as previously described (21, 55). The values of blank were subtracted from each value, and the viability values of the treated samples relative to the controls with vehicle were calculated. The values for the plots are the means±standard deviation.

Hemolytic Assay. The hemolytic activities of the newly synthesized compounds were determined and quantified as the amount of hemoglobin released by lysing porcine erythrocytes. Fresh blood drawn from the antecubital vein of a pig (Animal Care and Use Program, University of Manitoba) was centrifuged at 1000 g at 4° C. for 10 mins, washed with PBS thrice and resuspended in the same buffer. The final cell concentration used was 3×10⁸ cells/mL. Compounds were serially diluted with PBS and added to wells in a 96-well plate at twice the desired concentrations. Equal volumes of erythrocyte solution were then added to each well and incubated at 37° C. for 1 h. Intact erythrocytes were subsequently pelleted by centrifuging at 1000 g at 4° C. for 10 mins, and the supernatants were transferred to a new 96-well plate. Hemoglobin release was determined by measuring the absorbance on EMax® Plus microplate reader (Molecular Devices, Sunnyvale, Calif., USA) at 570 nm. Blood cells in PBS (0% hemolysis) and 0.1% Triton X-100 (100% hemolysis) were used as negative and positive controls, respectively. Percent hemolysis was calculated as [% hemolysis=(X−0%)/(100%−0%)], where X is the optical density values of the compounds at different concentrations (49).

Time-kill Assay. Time-kill curve analyses were performed by diluting a 30 μl aliquot of 0.5 McFarland standard of overnight culture to 3 ml of LB broth (containing novobiocin alone, and in combination with compound 1) and incubated at 37° C. with shaking at 250 rpm. At specific time intervals (0, 1, 3, 6, 9, and 24 h), 100 μl was taken from each sample, serially diluted in sterile PBS, plated on LB agar plates, and incubated at 37° C. in a humid 5% CO₂-enriched atmosphere. Bacterial colonies were counted after 20 h of incubation.

Outer Membrane Permeabilization Assay. The ability of the newly synthesized and reference compounds (compound 1, PMBN and tobramycin) to permeabilize the outer membrane of P. aeruginosa PAO1 was assessed using the nonpolar membrane-impermeable fluorescent probe 1-N-phenylnapthylamine (NPN) (40). Briefly, an overnight grown P. aeruginosa PAO1 culture was subcultured (1 in 100) in fresh LB broth and grown to a mid-logarithmic phase (approximately 2 h, OD₆₀₀=0.5-0.6). The cells were harvested by centrifuging for 10 min at 1000 g and room temperature, washed twice in PBS, and resuspended in half volume of PBS. This suspension was used in standard microtiter plate assay. Care was taken not to cool the suspension at any stage, since cooling the bacterial suspension below room temperature (i.e. during refrigerated centrifugation) could cause considerable increase in initial NPN uptake levels (40). To a black 96-well plate containing the cell culture was added NPN (10 μM final concentration), alone and in combination with various concentrations of test compounds. The resulting change in NPN fluorescence was measured immediately and continuously (every 30 secs) for 10 mins, with intermittent shaking, on a FlexStation 3 (Molecular Devices, Sunnyvale, USA) microplate reader at an excitation wavelength of 350 nm and emission wavelength of 420 nm. PMBN, a known outer membrane permeabilizer, served as a positive control while cells +NPN (without test compounds) served as a negative control. Four independent replicates were conducted, and the data were corrected for any background fluorescence.

Development of Resistance Study. The ability of novobiocin+compound 1 to suppress resistance development was determined by serial passaging, as previously described (19, 56). Briefly, wild-type A. baumannii ATCC 17978 cells were grown in 1 mL MHB media containing novobiocin (at ¼ MIC, ½ MIC, 1×MIC, 2×MIC, and 4×MIC), alone and in combination with compound 1. Tobramycin and colistin were included as controls. At 24-hour intervals, the cultures were assessed for growth. Cultures from the second highest concentrations that allowed visible growth were harvested and diluted to 0.5 McFarland in sterile PBS, followed by 1:50 dilution into fresh MHB media containing ¼ MIC, ½ MIC, 1×MIC, 2×MIC, and 4×MIC of each antibiotic. This serial passaging was repeated for 8 days. For novobiocin/compound 1 combination, the concentration of compound 1 was kept constant at 7.1 μM throughout the experiment. For cultures that grew at higher than the MIC levels, cultures in highest concentrations that permit growth were passaged on drug-free LB plates and MICs were determined by microbroth dilution in MHB.

Galleria mellonella In vivo Larvae-Infection Model. In vivo synergistic effects were determined using Galleria mellonella infection model, as previously described (49). Briefly, larvae were purchased from The Worm Lady Live Feeder (ON, Canada), stored in their natural habitat at 16° C., and used within 10 days of delivery. The larvae (average weight of 250 mg) were used for tolerability and efficacy studies. Tolerability study was performed by injecting a 10 μL aliquot of antimicrobial agents only at concentrations equivalent to 100 mg/kg or 200 mg/kg. The larvae (ten in each group) were incubated at 37° C. and monitored for 96 h. For efficacy studies, the virulence and bacterial load required to kill 100% of the larvae within 24 h (with no treatment) was first determined, which is approximately 10 CFU. Overnight grown culture of respective MDR A. baumannii isolate was standardized to 0.5 McFarland standard and diluted in PBS to a final concentration of 103 CFU/mL. A 10 μL aliquot of this solution (˜10 CFU) was injected into each larva and incubated for 3 h at 37° C. After the 3 h challenge, larvae in monotherapy experimental groups (fifteen per group) were treated with a 10 μL aliquot solution (containing different concentrations) of novobiocin, PMBN, compound 1, or PBS alone. The larvae in combination therapy groups were treated with novobiocin+compound 1 (25+25 mg/kg or 50+50 mg/kg) and novobiocin+PMBN (50+25 mg/kg). Larvae treated with 10 μL of PBS or high concentrations of test antibiotics served as negative and positive control, respectively. The larvae were incubated at 37° C. in Petri dishes lined with filter paper and scored for survivability every 6 h for up to 36 h. This experiment was repeated to give a total of 30 larvae in each case. Survival data curves were plotted using Kaplan-Meier survival analysis. Larvae were considered dead if they do not respond to touch.

While the preferred embodiments of the invention have been described above, it will be recognized and understood that various modifications may be made therein, and the appended claims are intended to cover all such modifications which may fall within the spirit and scope of the invention.

### REFERENCES

(1) Laxminarayan, R.; Duse, A.; Wattal, C.; Zaidi, A. K. M.; Wertheim, H. F. L.; Sumpradit, N.; Vlieghe, E.; Hara, G. L.; Gould, I. M.; Goossens, H.; Greko, C.; So, A. D.; Bigdeli, M.; Tomson, G.; Woodhouse, W.; Ombaka, E.; Peralta, A. Q.; Qamar, F. N.; Mir, F.; Kariuki, S.; Bhutta, Z. A.; Coates, A.; Bergstrom, R.; Wright, G. D.; Brown, E. D.; Cars, O. Antibiotic Resistance—the Need for Global Solutions. Lancet. Infect. Dis. 2013, 13, 1057-1098.
(2) Li, X.-Z.; Pl\tilde{e}siat, P.; Nikaido, H. The Challenge of Efflux-Mediated Antibiotic Resistance in Gram-Negative Bacteria. Clin. Microbiol. Rev. 2015, 28, 337-418.
(3) Nikaido, H. Molecular Basis of Bacterial Outer Membrane Permeability Revisited. Microbiol. Mol. Biol. Rev. 2003, 67, 593-656.
(4) Sherman, D. J.; Xie, R.; Taylor, R. J.; George, A. H.; Okuda, S.; Foster, P. J.; Needleman, D. J.; Kahne, D. Lipopolysaccharide Is Transported to the Cell Surface by a Membrane-to-Membrane Protein Bridge. Science. 2018, 359 (6377), 798-801.
(5) Vaara, M. Agents That Increase the Permeability of the Outer Membrane. Microbiol. Rev. 1992, 56, 395-411.
(6) Lin, L.; Tan, B.; Pantapalangkoor, P.; Ho, T.; Baquir, B.; Tomaras, A.; Montgomery, J. I.; Reilly, U.; Barbacci, E. G.; Hujer, K.; Bonomo, R. A.; Fernandez, L.; Hancock, R. E. W.; Adams, M. D.; French, S. W.; Buslon, V. S.; Spellberg, B. Inhibition of LpxC Protects Mice from Resistant Acinetobacter baumannii by Modulating Inflammation and Enhancing Phagocytosis. MBio 2012, 3, e00312-12.
(7) May, J. M.; Owens, T. W.; Mandler, M. D.; Simpson, B. W.; Lazarus, M. B.; Sherman, D. J.; Davis, R. M.; Okuda, S.; Massefski, W.; Ruiz, N.; Kahne, D. The Antibiotic Novobiocin Binds and Activates the ATPase That Powers Lipopolysaccharide Transport. J. Am. Chem. Soc. 2017, 139, 17221-17224.
(8) Viljanen, P.; Vaara, M. Susceptibility of Gram-Negative Bacteria to Polymyxin B Nonapeptide. Antimicrob. Agents Chemother. 1984, 25 (6), 701-705.
(9) Corbett, D.; Wise, A.; Langley, T.; Skinner, K.; Trimby, E.; Birchall, S.; Dorali, A.; Sandiford, S.; Williams, J.; Warn, P.; Vaara, M.; Lister, T. Potentiation of Antibiotic Activity by a Novel Cationic Peptide: Potency and Spectrum of Activity of SPR741. Antimicrob. Agents Chemother. 2017, 61, e00200-17.
(10) Zabawa, T. P.; Pucci, M. J.; Parr, T. R.; Lister, T. Treatment of Gram-Negative Bacterial Infections by Potentiation of Antibiotics. Curr. Opin. Microbiol. 2016, 33, 7-12.
(11) Herzog, I. M.; Green, K. D.; Berkov-Zrihen, Y.; Feldman, M.; Vidavski, R. R.; Eldar-Boock, A.; Satchi-Fainaro, R.; Eldar, A.; Garneau-Tsodikova, S.; Fridman, M. 6″-Thioether Tobramycin Analogues: Towards Selective Targeting of Bacterial Membranes. Angew. Chemie—Int. Ed. 2012, 51, 5652-5656.
(12) Zimmermann, L.; Bussibre, A.; Ouberai, M.; Baussanne, I.; Jolivalt, C.; Mingeot-Leclercq, M.-P.; Décout, J.-L. Tuning the Antibacterial Activity of Amphiphilic Neamine Derivatives and Comparison to Paromamine Homologues. J. Med. Chem. 2013, 56, 7691-7705.
(13) Gorityala, B. K.; Guchhait, G.; Fernando, D. M.; Deo, S.; McKenna, S. A.; Zhanel, G. G.; Kumar, A.; Schweizer, F. Adjuvants Based on Hybrid Antibiotics Overcome Resistance in Pseudomonas aeruginosa and Enhance Fluoroquinolone Efficacy. Angew. Chem. Int. Ed. Engl. 2016, 55, 555-559.
(14) Domalaon, R.; Idowu, T.; Zhanel, G. G.; Schweizer, F. Antibiotic Hybrids: The Next Generation of Agents and Adjuvants against Gram-Negative Pathogens? Clin. Microbiol. Rev. 2018, 31, e00077-17.
(15) Böttger, E. C.; Springer, B.; Prammananan, T.; Kidan, Y.; Sander, P. Structural Basis for Selectivity and Toxicity of Ribosomal Antibiotics. EMBO Rep. 2001, 2, 318-323.
(16) Hobbie, S. N.; Akshay, S.; Kalapala, S. K.; Bruell, C. M.; Shcherbakov, D.; Bottger, E. C. Genetic Analysis of Interactions with Eukaryotic RRNA Identify the Mitoribosome as Target in Aminoglycoside Ototoxicity. Proc. Natl. Acad. Sci. 2008, 105, 20888-20893.
(17) Matt, T.; Ng, C. L.; Lang, K.; Sha, S.-H.; Akbergenov, R.; Shcherbakov, D.; Meyer, M.; Duscha, S.; Xie, J.; Dubbaka, S. R.; Perez-Fernandez, D.; Vasella, A.; Ramakrishnan, V.; Schacht, J.; Böttger, E. C. Dissociation of Antibacterial Activity and Aminoglycoside Ototoxicity in the 4-Monosubstituted 2-Deoxystreptamine Apramycin. Proc. Natl. Acad. Sci. 2012, 109, 10984-10989.
(18) Shulman, E.; Belakhov, V.; Wei, G.; Kendall, A.; Meyron-Holtz, E. G.; Ben-Shachar, D.; Schacht, J.; Baasov, T. Designer Aminoglycosides That Selectively Inhibit Cytoplasmic Rather than Mitochondrial Ribosomes Show Decreased Ototoxicity: A Strategy for the Treatment of Genetic Diseases. J. Biol. Chem. 2014, 289, 2318-2330.
(19) Lyu, Y.; Yang, X.; Goswami, S.; Gorityala, B. K.; Idowu, T.; Domalaon, R.; Zhanel, G. G.; Shan, A.; Schweizer, F. Amphiphilic Tobramycin-lysine Conjugates Sensitize Multidrug Resistant Gram-Negative Bacteria to Rifampicin and Minocycline. J. Med. Chem. 2017, 60, 3684-3702.
(20) Herzog, I. M.; Feldman, M.; Eldar-Boock, A.; Satchi-Fainaro, R.; Fridman, M. Design of Membrane Targeting Tobramycin-Based Cationic Amphiphiles with Reduced Hemolytic Activity. Med. Chem. Commun. 2013, 4, 120-124.
(21) Idowu, T.; Samadder, P.; Arthur, G.; Schweizer, F. Amphiphilic Modulation of Glycosylated Antitumor Ether Lipids Results in a Potent Triamino Scaffold against Epithelial Cancer Cell Lines and BT474 Cancer Stem Cells. J. Med. Chem. 2017, 60, 9724-9738.
(22) Findlay, B.; Zhanel, G. G.; Schweizer, F. Cationic Amphiphiles, a New Generation of Antimicrobials Inspired by the Natural Antimicrobial Peptide Scaffold. Antimicrob. Agents Chemother. 2010, 54, 4049-4058.
(23) Fed. Regist. 2011, 76 (12), 3134.
(24) Murren, J. R.; DiStasio, S. A.; Lorico, A.; McKeon, A.; Zuhowski, E. G.; Egorin, M. J.; Sartorelli, A. C.; Rappa, G. Phase I and Pharmacokinetic Study of Novobiocin in Combination with VP-16 in Patients with Refractory Malignancies. Cancer J. 2000, 6, 256-265.
(25) Hahm, H. A.; Armstrong, D. K.; Chen, T. L.; Grochow, L.; Passos-Coelho, J.; Goodman, S. N.; Davidson, N. E.; Kennedy, M. J. Novobiocin in Combination with High-Dose Chemotherapy for the Treatment of Advanced Breast Cancer: A Phase 2 Study. Biol. blood marrow Transplant. 2000, 6, 335-343.
(26) Ofek, I.; Cohen, S.; Rahmani, R.; Kabha, K.; Tamarkin, D.; Herzig, Y.; Rubinstein, E. Antibacterial Synergism of Polymyxin B Nonapeptide and Hydrophobic Antibiotics in Experimental Gram-Negative Infections in Mice. Antimicrob. Agents Chemother. 1994, 38, 374-377.
(27) Tsubery, H.; Ofek, I.; Cohen, S.; Fridkin, M. Structure-Function Studies of Polymyxin B Nonapeptide: Implications to Sensitization of Gram-Negative Bacteria. J. Med. Chem. 2000, 43, 3085-092.
(28) Berkov-Zrihen, Y.; Green, K. D.; Labby, K. J.; Feldman, M.; Garneau-Tsodikova, S.; Fridman, M. Synthesis and Evaluation of Hetero- and Homodimers of Ribosome-Targeting Antibiotics: Antimicrobial Activity, in Vitro Inhibition of Translation, and Drug Resistance. J. Med. Chem. 2013, 56, 5613-5625.
(29) Tyers, M.; Wright, G. D. Drug Combinations: A Strategy to Extend the Life of Antibiotics in the 21st Century. Nat. Rev. Microbiol. 2019, 17, 141-155.
(30) Breidenstein, E. B. M.; de la Fuente-Nunez, C.; Hancock, R. E. W. 15 Pseudomonas aeruginosa: All Roads Lead to Resistance. Trends Microbiol. 2011, 19, 419-426.
(31) Richter, M. F.; Drown, B. S.; Riley, A. P.; Garcia, A.; Shirai, T.; Svec, R. L.; Hergenrother, P. J. Predictive Compound Accumulation Rules Yield a Broad-Spectrum Antibiotic. Nature 2017, 545, 299-304.
(32) Zhanel, G. G.; Lawson, C. D.; Zelenitsky, S.; Findlay, B.; Schweizer, F.; Adam, H.; Walkty, A.; Rubinstein, E.; Gin, A. S.; Hoban, D. J.; Lynch, J. P.; Karlowsky, J. A.

Comparison of the Next-Generation Aminoglycoside Plazomicin to Gentamicin, Tobramycin and Amikacin. Expert Rev. Anti. Infect. Ther. 2012, 10, 459-473.
(33) Pai, M. P.; Nafziger, A. N.; Bertino, J. S. Simplified Estimation of Aminoglycoside Pharmacokinetics in Underweight and Obese Adult Patients. Antimicrob. Agents Chemother. 2011, 55, 4006-4011.
(34) Stokes, J. M.; Macnair, C. R.; Ilyas, B.; French, S.; Côté, J.-P.; Bouwman, C.; Farha, M. A.; Sieron, A. O.; Whitfield, C.; Coombes, B. K.; Brown, E. D. Pentamidine Sensitizes Gram-Negative Pathogens to Antibiotics and Overcomes Acquired Colistin Resistance. Nat. Microbiol. 2017, 2, 17028.
(35) Liu, Y.-Y.; Wang, Y.; Walsh, T. R.; Yi, L.-X.; Zhang, R.; Spencer, J.; Doi, Y.; Tian, G.; Dong, B.; Huang, X.; Yu, L-F.; Gu, D.; Ren, H.; Chen, X.; Lv, L.; He, D.; Zhou, H.; Liang, Z.; Liu, J-H.; Shen, J. Emergence of Plasmid-Mediated Colistin Resistance Mechanism MCR-1 in Animals and Human Beings in China: A Microbiological and Molecular Biological Study. Lancet Infect. Dis. 2015, 16, 161-168.
(36) Taylor, P. L.; Rossi, L.; De Pascale, G.; Wright, G. D. A Forward Chemical Screen Identifies Antibiotic Adjuvants in Escherichia coli. ACS Chem. Biol. 2012, 7, 1547-1555.
(37) French, P.; Venuti, E.; Fraimow, H. S. In Vitro Activity of Novobiocin against Multiresistant Strains of Enterococcus faecium. Antimicrob. Agents Chemother. 1993, 37, 2736-2739.
(38) Lomovskaya, O.; Warren, M. S.; Lee, A.; Fronko, R.; Lee, M.; Blais, J.; Chamberland, S.; Renau, T.; Leger, R.; Hecker, S.; Watkins, W.; Hoshino, K.; Ishida, H.; Lee, V. J. Identification and Characterization of Inhibitors of Multidrug Resistance Efflux Pumps in Pseudomonas aeruginosa: Novel Agents for Combination Therapy. Antimicrob. Agents Chemother. 2001, 45, 105-116.
(39) Kumar, A.; Chua, K. L.; Schweizer, H. P. Method for Regulated Expression of Single-Copy Efflux Pump Genes in a Surrogate Pseudomonas aeruginosa Strain: Identification of the BpeEF-OprC Chloramphenicol and Trimethoprim Efflux Pump of Burkholderia pseudomallei 1026b. Antimicrob. Agents Chemother. 2006, 50, 3460-3463.
(40) Helander, I. M.; Mattila-Sandholm, T. Fluorometric Assessment of Gram-Negative Bacterial Permeabilization. J. Appl. Microbiol. 2000, 88, 213-219.
(41) Bisacchi, G. S.; Manchester, J. I. A New-Class Antibacterial—Almost. Lessons in Drug Discovery and Development: A Critical Analysis of More than 50 Years of Effort toward ATPase Inhibitors of DNA Gyrase and Topoisomerase IV. ACS Infect. Dis. 2014, 1, 4-41.
(42) Moffatt, J. H.; Harper, M.; Harrison, P.; Hale, J. D. F.; Vinogradov, E.; Seemann, T.; Henry, R.; Crane, B.; St Michael, F.; Cox, A. D.; Adler, B.; Nation, R. L.; Lian, J.; Boyce, J. D. Colistin Resistance in Acinetobacter baumannii Is Mediated by Complete Loss of Lipopolysaccharide Production. Antimicrob. Agents Chemother. 2010, 54, 4971-4977.
(43) Adams, M. D.; Nickel, G. C.; Bajaksouzian, S.; Lavender, H.; Murthy, A. R.; Jacobs, M. R.; Bonomo, R. A. Resistance to Colistin in Acinetobacter baumannii Associated with Mutations in the PmrAB Two-Component System. Antimicrob. Agents Chemother. 2009, 53, 3628-3634.
(44) Mandler, M. D.; Baidin, V.; Lee, J.; Pahil, K. S.; Owens, T. W.; Kahne, D. Novobiocin Enhances Polymyxin Activity by Stimulating Lipopolysaccharide Transport. J. Am. Chem. Soc. 2018, 140, 6749-6753.
(45) Stanger, F. V.; Dehio, C.; Schirmer, T. Structure of the N-Terminal Gyrase B Fragment in Complex with ADP-Pi Reveals Rigid-Body Motion Induced by ATP Hydrolysis. PLoS One 2014, 9, e107289.
(46) Rita Brochado, A.; Telzerow, A.; Bobonis, J.; Banzhaf, M.; Mateus, A.; Selkrig, J.; Huth, E.; Bassler, S.; Beas, J. Z.; Zietek, M.; Ng, N.; Foerster, S.; Ezraty, B.; Py, B.; Barras, F.; Savitski, M. M.; Göttig, S.; Athanasios T. Species-Specific Activity of Antibacterial Drug Combinations. Nature 2018, 559, 259-263.
(47) Hill, L.; Veli, N.; Coote, P. J. Evaluation of Galleria mellonella Larvae for Measuring the Efficacy and Pharmacokinetics of Antibiotic Therapies against Pseudomonas aeruginosa Infection. Int. J. Antimicrob. Agents 2014, 43, 254-261.
(48) Idowu, T.; Ammeter, D.; Arthur, G.; Zhanel, G. G.; Schweizer, F. Potentiation of β-Lactam Antibiotics and β-Lactam/β-Lactamase Inhibitor Combinations against Multidrug and Extensively Drug-Resistant Pseudomonas aeruginosa Using Non-Ribosomal Tobramycin-Cyclam Conjugates. J. Antimicrob. Chemother. 2019, 74, 2640-2648
(49) Idowu, T.; Arthur, G.; Zhanel, G. G.; Schweizer, F. Heterodimeric Rifampicin-Tobramycin Conjugates Break Intrinsic Resistance of Pseudomonas aeruginosa to Doxycycline and Chloramphenicol In Vitro and in a Galleria mellonella In Vivo Model. Eur. J. Med. Chem. 2019, 174, 16-32.
(50) Zhanel, G. G.; DeCorby, M.; Laing, N.; Weshnoweski, B.; Vashisht, R.; Tailor, F.; Nichol, K. A.; Wierzbowski, A.; Baudry, P. J.; Karlowsky, J. A.; Lagacé-Wiens, P.; Walkty, A.; McCracken, M.; Mulvey, M. R.; Johnson, J.; Hoban, D. J. Antimicrobial-Resistant Pathogens in Intensive Care Units in Canada: Results of the Canadian National Intensive Care Unit (CAN-ICU) Study, 2005-2006. Antimicrob. Agents Chemother. 2008, 52, 1430-1437.
(51) Zhanel, G. G.; Adam, H. J.; Baxter, M. R.; Fuller, J.; Nichol, K. A.; Denisuik, A. J.; Lagacé-Wiens, P. R. S.; Walkty, A.; Karlowsky, J. A.; Schweizer, F.; Hoba, D. J. Antimicrobial Susceptibility of 22746 Pathogens from Canadian Hospitals: Results of the CANWARD 2007-11 Study. J. Antimicrob. Chemother. 2013, 68 Suppl 1, 17-22.
(52) Zhanel, G. G.; DeCorby, M.; Adam, H.; Mulvey, M. R.; McCracken, M.; Lagacé-Wiens, P.; Nichol, K. A.; Wierzbowski, A.; Baudry, P. J.; Tailor, F.; Karlowsky, J. A.; Walkty, A.; Schweizer, F.; Johnson, J.; Hoban, D. J. Prevalence of Antimicrobial-Resistant Pathogens in Canadian Hospitals: Results of the Canadian Ward Surveillance Study (CANWARD 2008). Antimicrob. Agents Chemother. 2010, 54, 4684-4693.
(53) Magiorakos, A. P.; Srinivasan, A.; Carey, R. B.; Carmeli, Y.; Falagas, M. E.; Giske, C. G.; Harbarth, S.; Hindler, J. F.; Kahlmeter, G.; Olsson-Liljequist, B.; Paterson, D. L.; Rice, L. B.; Stelling, J.; Struelens, M. J.; Vatopoulos, A.; Weber, J. T.; Monnet, D. L. Multidrug-Resistant, Extensively Drug-Resistant and Pandrug-Resistant Bacteria: An International Expert Proposal for Interim Standard Definitions for Acquired Resistance. Clin. Microbiol. Infect. 2012, 18, 268-281.
(54) Clinical and Laboratory Standards Institute. 2017. Performance Standards for Antimicrobial Susceptibility Testing. Twenty-seventh Informational Supplement. CLSI document M100-S27. CLSI, Wayne, Pa.
(55) Idowu, T.; Samadder, P.; Arthur, G.; Schweizer, F. Design, Synthesis and Antitumor Properties of Glycosylated Antitumor Ether Lipid (GAEL)-Chlorambucil-Hybrids. Chem. Phys. Lipids 2016, 194, 139-148.
(56) Ling, L. L.; Schneider, T.; Peoples, A. J.; Spoering, A. L.; Engels, I.; Conlon, B. P.; Mueller, A.; Schäberle, T. F.; Hughes, D. E.; Epstein, S.; Jones, M.; Lazarides, L.; Steadman, V. A.; Cohen, D. R.; Felix, C. R.; Fetterman, K. A.; Millett, W. P.; Nitti, A. G.; Zullo, A. M.; Chen, C.; Lewis, K. A New Antibiotic Kills Pathogens without Detectable Resistance. Nature 2015, 517, 455-459.
(57) Bush, K.; Bradford, P. A. β-Lactams and β-lactamase inhibitors: an overview. Cold Spring Harb. Perspect. Med. 2016, 6, a025247.
(58) Winkler, M. L.; Papp-Wallace, K. M.; Hujer, A. M.; Domitrovic, T. N.; Hujer, K. M.; Hurless, K. N.; Tuohy, M.; Hall, G.; Bonomo, R. A. Unexpected challenges in treating multidrug-resistant Gram-negative bacteria: resistance to ceftazidime-avibactam in archived isolates of Pseudomonas aeruginosa. Antimicrob. Agents Chemother. 2015, 59, 1020-1029.
(59) Ammeter, D.; Idowu, T.; Zhanel, G. G.; Schweizer, F. Development of a nebramine-cyclam conjugate as an antibacterial adjuvant to potentiate β-lactam antibiotics against multidrug-resistant P. aeruginosa. J. Antibiot. (Tokyo). 2019, 72, 816-826.
(60) Idowu, T.; Ammeter, D.; Rossong, H.; Zhanel, G. G.; Schweizer, F. Homodimeric tobramycin adjuvant repurposes novobiocin as an effective antibacterial agent against Gram-negative bacteria. J. Med. Chem. 2019, 62, 9103-9115.
(61) Yang, X.; Ammeter, D.; Idowu, T.; Domalaon, R.; Brizuela, M.; Okunnu, O.; Bi, L.; Guerrero, Y. A.; Zhanel, G. G.; Kumar, A.; et al. Amphiphilic nebramine-based hybrids rescue legacy antibiotics from intrinsic resistance in multidrug-resistant Gram-negative bacilli. Eur. J. Med. Chem. 2019, 175, 187-200.
(62) Conejo, M. C.; Garcia, I.; Martínez-Martfnez, L.; Picabea, L.; Pascual, A. Zinc eluted from siliconized latex urinary catheters decreases OprD expression, causing carbapenem resistance in Pseudomonas aeruginosa. Antimicrob. Agents Chemother. 2003, 47, 2313-5.
(63) Perron, K.; Caille, O.; Rossier, C.; van Delden, C.; Dumas, J.-L.; Kohler, T. CzcR-CzcS, a two-component system involved in heavy metal and carbapenem resistance in Pseudomonas aeruginosa. J. Biol. Chem. 2004, 279, 8761-8768.
(64) Caille, O.; Rossier, C.; Perron, K. A copper-activated two-component system interacts with zinc and imipenem resistance in Pseudomonas aeruginosa. J. Bacteriol. 2007, 189, 4561-8.
(65) Idowu, T.; Schweizer, F. Ubiquitous nature of fluoroquinolones: the oscillation between antibacterial and anticancer activities. Antibiotics 2017, 6, 26.
(66) Masuda, N.; Sakagawa, E.; Ohya, S.; Gotoh, N.; Tsujimoto, H.; Nishino, T. Contribution of the MexX-MexY-oprM efflux system to intrinsic resistance in Pseudomonas aeruginosa. Antimicrob. Agents Chemother. 2000, 44, 2242-6.
(67) Masuda, N.; Sakagawa, E.; Ohya, S.; Gotoh, N.; Tsujimoto, H.; Nishino, T. Substrate specificities of MexAB-OprM, MexCD-OprJ, and MexXY-oprM efflux pumps in Pseudomonas aeruginosa. Antimicrob. Agents Chemother. 2000, 44, 3322-7.
(68) Nakamura, A.; Hosoda, M.; Kato, T.; Yamada, Y.; Itoh, M.; Kanazawa, K.; Nouda, H. Combined effects of meropenem and aminoglycosides on Pseudomonas aeruginosa in vitro. J. Antimicrob. Chemother. 2000, 46, 901-904.
(69) So, W.; Shurko, J.; Galega, R.; Quilitz, R.; Greene, J. N.; Lee, G. C. Mechanisms of high-level ceftolozane/tazobactam resistance in Pseudomonas aeruginosa from a severely neutropenic patient and treatment success from synergy with tobramycin. J. Antimicrob. Chemother. 2019, 74, 269-271.
(70) Stover, C. K.; Pham, X. Q.; Erwin, A. L.; Mizoguchi, S. D.; Warrener, P.; Hickey, M. J.; Brinkman, F. S.; Hufnagle, W. O.; Kowalik, D. J.; Lagrou, M.; et al. Complete genome sequence of Pseudomonas aeruginosa PAO1, an opportunistic pathogen. Nature 2000, 406, 959-64.
(71) Lister, P. D.; Wolter, D. J.; Hanson, N. D. Antibacterial-resistant Pseudomonas aeruginosa: clinical impact and complex regulation of chromosomally encoded resistance mechanisms. Clin. Microbiol. Rev. 2009, 22, 582-610.
(72) Ortiz de la Rosa, J.-M.; Nordmann, P.; Poirel, L. ESBLs and resistance to ceftazidime/avibactam and ceftolozane/tazobactam combinations in Escherichia coli and Pseudomonas aeruginosa. J. Antimicrob. Chemother. 2019, 74, 1934-1939.
(73) Haidar, G.; Philips, N. J.; Shields, R. K.; Snyder, D.; Cheng, S.; Potoski, B. A.; Doi, Y.; Hao, B.; Press, E. G.; Cooper, V. S.; et al. Ceftolozane-tazobactam for the treatment of multidrug-resistant Pseudomonas aeruginosa infections: clinical effectiveness and evolution of resistance. Clin. Infect. Dis. 2017, 65, 110-120.
(74) Queenan, A. M.; Bush, K. Carbapenemases: The versatile β-lactamases. Clin. Microbiol. Rev. 2007, 20, 440-458.
(75) El Amin, N.; Giske, C. G.; Jalal, S.; Keijser, B.; Kronvall, G.; Wretlind, B. Carbapenem resistance mechanisms in Pseudomonas aeruginosa: alterations of porin OprD and efflux proteins do not fully explain resistance patterns observed in clinical isolates. APMIS 2005, 113, 187-196.
(76) Zhanel, G. G.; Wiebe, R.; Dilay, L.; Thomson, K.; Rubinstein, E.; Hoban, D. J.; Noreddin, A. M.; Karlowsky, J. A. Comparative review of the carbapenems. Drugs 2007, 67, 1027-1052.

TABLE 1
Tobramycin homodimer potentiates novobiocin against Gram-negative bacteria.
Synergistic effects of novobiocin and compound 1 (at ≤7.1 μM,
i.e. ≤16 μg/ml) against wild-type and clinical isolates of Gram-
negative bacteria. MIC of compound 1 is >128 μg/ml against all strains.

| | | MIC (μg/ml) of Novobiocin | | |
|---|---|---|---|---|
| Bacteria | Strain | Alone | +Compound 1 | Fold Potentiation |
| P. aeruginosa | PAO1^{†} | 512 | 4 | 128 |
| | 259-96918^{‡,} ^{§} | 512 | 8 | 64 |
| | 260-97103^{‡,} ^{§} | 256 | 1 | 256 |
| | 262-101856^{‡, §} | 512 | 8 | 64 |
| | 264-104354^{‡, §} | 512 | 8 | 64 |
| | 91433^{‡, §,} * | 16 | 1 | 16 |
| | 100036^{‡, §} | 1024 | 8 | 128 |
| | 101243^{‡, §,} * | 128 | 1 | 128 |
| | 101885^{‡, §} | 1024 | 8 | 128 |
| | 114228^{‡, §,} * | 512 | 16 | 32 |
| A. baumannii | ATCC 17978^{†} | 16 | 0.5 | 32 |
| | 027^{‡} | 8 | 0.5 | 16 |
| | 031^{‡} | 8 | 0.5 | 16 |
| | 92247^{‡,} * | 4 | 0.25 | 16 |
| | 110193^{‡} | 256 | 1 | 256 |
| | LAC-4^{‡} | 8 | 0.5 | 16 |
| E. coli | ATCC 25922^{†} | 64 | 2 | 32 |
| | 94393^{‡,} *^{#} | 128 | 2 | 64 |
| | 94474^{‡,} *^{#} | 256 | 16 | 16 |
| | 107115^{‡, §} | 128 | 8 | 16 |
| K. pneumoniae | 113250^{‡,} * | 512 | 16 | 32 |
| | 113254^{‡,} * | 256 | 16 | 16 |
| | 116381^{‡} | 1024 | 8 | 128 |
| E. cloacae | 117029^{‡} | 1024 | 8 | 128 |
| | 118568^{‡,} * | 1024 | 32 | 32 |
| | 121187^{‡,} * | 64 | 8 | 8 |

^{†}Wildtype;
^{‡}Clinical isolate;
^{§}Carbapenem-resistant;
*Colistin resistant;
^{#}mcr-1 gene positive.

TABLE 2
Compound 1 potentiates Novobiocin better than PMBN
against colistin-resistant Gram-negative bacteria.

| | MIC (μg/mL) of Novobiocin | | |
|---|---|---|---|
| Organism | Alone | +PMBN (7.1 μM) | +1 (7.1 μM) |
| PAO1^{†} | 512 | 4 | 4 |
| PA 91433^{‡} | 16 | 4 | 1 |
| PA 114228^{‡} | 512 | 128 | 16 |
| PA 101243^{‡} | 128 | 8 | 1 |
| E. coli 94393^{‡} | 128 | 4 | 2 |
| E. coli 94474^{‡} | 256 | 32 | 16 |
| AB 92247^{‡} | 4 | 1 | 0.25 |
| KP 113254^{‡} | 256 | 64 | 16 |
| EC 118568^{‡} | 1024 | 512 | 32 |

PA = P. aeruginosa; AB = A. baumannii; KP = K. pneumoniae; EC = E. cloacae.
^{†}Wild type;
^{‡}Clinical isolate.

TABLE 3
Potentiation of novobiocin in P. aeruginosa by
compound 1 is independent of RND efflux pumps.

| | | MIC of Novobiocin | | |
|---|---|---|---|---|
| | MIC of | | +7.1 μM | |
| Strain | Compound 1 | Alone | Compound 1 | Fold Potentiation |
| PAO1 | >128 | 512 | 4 | 128 |
| PAO200 | >128 | 32 | 0.125 | 256 |
| PAO750 | >128 | 32 | 0.125 | 256 |

PAO1 = wild-type, PAO200 (ΔmexAB-oprM) and PAO750 (ΔmexAB-oprM, ΔmexCD-oprJ, ΔmexEF-oprN, ΔmexJK, ΔmexXY, and ΔopmH outer membrane) are efflux-deficient strains.
^{38, 39} MICs are reported in μg/ml.

TABLE 4
Susceptibility profiles (MIC in μg/ml) of wild-type
A. baumannii ATCC 17978 versus resistant mutants generated
from seven serial passages (Day 8) of exposure to sub-MICs
novobiocin (Mutant A), colistin (Mutant B) and novobiocin
+ 7.1 μM compound 1 (Mutant C).

| Antibiotics | Wild-type | Mutant A | Mutant B | Mutant C |
|---|---|---|---|---|
| Novobiocin | 16 | 2048 | <0.031 | 128 |
| Tobramycin | 1 | 1 | 0.25 | 1 |
| Ciprofloxacin | 1 | 16 | 0.0625 | 1 |
| Minocycline | 0.125 | 0.5 | <0.031 | 0.125 |
| Rifampicin | 2 | 2 | <0.031 | 2 |
| Ceftazidime | 16 | 32 | 1 | 16 |
| Chloramphenicol | 64 | 32 | 8 | 64 |
| Colistin | 0.063 | 8 | 1024 | 0.031 |
| Novobiocin + 1 | 0.5 | 128 | <0.031 | 16 |

TABLE 5
MICs (μg/ml) of tobramycin (TOB), ciprofloxacin (CIP)
and compounds 21-27 against wild-type P. aeruginosa PAO1
and efflux deficient mutants PAO200^{a} and PAO750^{b}.

| Compounds | PAO1 | PAO200 | PAO200 |
|---|---|---|---|
| TOB | 1 | 1 | 1 |
| CIP | 0.125 | 0.0156 | 0.0078 |
| 21 | 32 | 4 | 2 |
| 22 | >128 | >128 | 128 |
| 23 | >128 | >128 | >128 |
| 24 | 32 | 4 | 2 |
| 25 | >128 | >128 | >128 |
| 26 | >128 | >128 | 128 |
| 27 | 32 | 16 | 16 |

^{a}(ΔmexA-mexB-oprM) ^{b}(ΔmexAB-oprM, ΔmexCD-oprJ, ΔmexEF-oprN, Δmex JK, ΔmexXY, and ΔopmH).

TABLE 6
Compounds 21-27 (at 8 μM) potentiate β-lactam
antibiotics against P. aeruginosa PAO1.

| MIC | Aztreonam | Ceftazidime | Imipenem | Piperacillin |
|---|---|---|---|---|
| Alone | 4 | 2 | 4 | 8 |
| +compound 21 | 0.25 | 0.25 | 0.5 | 2 |
| +compound 22 | 0.25 | 0.25 | 2 | 2 |
| +compound 23 | 0.25 | 0.25 | 1 | 0.5 |
| +compound 24 | 0.5 | 0.25 | 1 | 2 |
| +compound 25 | 0.5 | 0.5 | 4 | 2 |
| +compound 26 | 1 | 0.5 | 2 | 8 |
| +compound 27 | 0.5 | 0.25 | 1 | 8 |

TABLE 7
Checkerboard studies between aztreonam (ATM) and compounds
21-27 (at 8 μM) against P. aeruginosa (PA) isolates.

| | | MIC of ATM in the presence of | | | | | | |
|---|---|---|---|---|---|---|---|---|
| Strain | ATM alone | 21 | 22^{a} | 23 | 24 | 25^{b} | 26 | 27 |
| PAO1 | 4 | 0.25 | 0.25 | 0.25 | 0.5 | 0.5 | 1 | 0.5 |
| PA 259-96918^{†, ‡} | 32 | 8 | 4 | 8 | 8 | 8 | 8 | 32 |
| PA 260-97103^{†} | 64 | 4 | 8 | 16 | 0.25 | 8 | 16 | 8 |
| PA 262-101856^{†} | 32 | 4 | 8 | 8 | 16 | 8 | 16 | 32 |
| PA 264-104354^{†} | 32 | 8 | 8 | 8 | 16 | 16 | 8 | 32 |
| PA 91433^{†,}* | 16 | 4 | 16 | 4 | 8 | 8 | 2 | 16 |
| PA 100036^{†} | 16 | 4 | 2 | 2 | 8 | 8 | 4 | 16 |
| PA 101885^{†} | 16 | 1 | 0.5 | 4 | 4 | 4 | 4 | 4 |
| PA 101243^{†,}* | 32 | 4 | 8 | 16 | 0.25 | 2 | 8 | 4 |
| PA 114228^{†,}* | 32 | 2 | 4 | 4 | 8 | 16 | 16 | 8 |
| PA 86052^{†} | 64 | 8 | 16 | 8 | 4 | 64 | 32 | 32 |
| PA 88949^{†} | 32 | 8 | 16 | 32 | 8 | 32 | 16 | 32 |
| PA 107092^{†} | 64 | 8 | 16 | 16 | 32 | 32 | 32 | 32 |
| PA 108590^{†} | 32 | 0.5 | 8 | 8 | 0.13 | 8 | 16 | 2 |
| PA 109084^{†} | 64 | 16 | 8 | 8 | 32 | 32 | 16 | 32 |
| PA O200^{#} | 0.25 | 0.03 | 0.02 | 0.03 | 0.03 | 0.06 | 0.06 | 0.06 |
| PA O750^{#} | 0.5 | 0.06 | 0.03 | 0.06 | 0.13 | 0.13 | 0.25 | 0.06 |

^{†}Clinical isolate;
^{‡}MBL-expressing;
*colistin-resistant;
^{#}efflux-deficient.
^{a}Previously published data [11]; ^{b}Previously published data [12].

TABLE 8
Checkerboard studies between ceftazidime (CAZ) and compounds
21-27 (at 8 μM) against P. aeruginosa (PA) isolates.

| | | MIC of CAZ in the presence of | | | | | | |
|---|---|---|---|---|---|---|---|---|
| Strain | CAZ alone | 21 | 22^{a} | 23 | 24 | 25^{b} | 26 | 27 |
| PAO1 | 2 | 0.25 | 0.25 | 0.25 | 0.25 | 0.5 | 0.5 | 0.25 |
| PA 259-96918^{†, ‡} | 512 | 64 | 64 | 128 | 128 | 128 | 128 | 512 |
| PA 260-97103^{†} | 32 | 4 | 16 | 8 | 0.25 | 16 | 16 | 16 |
| PA 262-101856^{†} | 16 | 4 | 4 | 2 | 4 | 16 | 16 | 16 |
| PA 264-104354^{†} | 128 | 16 | 32 | 32 | 64 | 32 | 32 | 128 |
| PA 91433^{†,}* | 4 | 2 | 2 | 1 | 0.5 | 2 | 0.5 | 4 |
| PA 100036^{†} | 8 | 2 | 2 | 2 | 4 | 4 | 4 | 6 |
| PA 101885^{†} | 8 | 1 | 1 | 2 | 4 | 2 | 1 | 2 |
| PA 101243^{†,}* | 64 | 8 | 8 | 64 | 4 | 8 | 32 | 16 |
| PA 114228^{†,}* | 8 | 0.5 | 1 | 1 | 4 | 4 | 1 | 2 |
| PA 86052^{†} | 64 | 32 | 64 | 64 | 4 | 64 | 64 | 32 |
| PA 88949^{†} | 64 | 32 | 16 | 16 | 32 | 64 | 64 | 32 |
| PA 107092^{†} | 128 | 16 | 32 | 32 | 64 | 32 | 128 | 32 |
| PA 108590^{†} | 32 | 0.5 | 8 | 8 | 1 | 16 | 32 | 2 |
| PA 109084^{†} | 64 | 16 | 16 | 32 | 32 | 32 | 32 | 32 |
| PA O200^{#} | 1 | 0.06 | 0.06 | 0.13 | 0.13 | 0.13 | 0.25 | 0.25 |
| PA O750^{#} | 0.5 | 0.03 | 0.06 | 0.06 | 0.01 | 0.13 | 0.13 | 0.25 |

^{†}Clinical isolate;
^{‡}MBL-expressing;
*colistin-resistant;
^{#}efflux-deficient.
Previously published data [11]; Previously published data [12].

TABLE 9
Checkerboard studies between imipenem (IPM) and compounds
21-27 (at 8 μM) against P. aeruginosa (PA) isolates.

| | | MIC of IPM in the presence of | | | | | | |
|---|---|---|---|---|---|---|---|---|
| Strain | IPM alone | 21 | 22^{a} | 23 | 24 | 25^{b} | 26 | 27 |
| PAO1 | 4 | 0.5 | 2 | 1 | 1 | 4 | 2 | 1 |
| PA 259-96918^{†, ‡} | 64 | 16 | 64 | 32 | 16 | 32 | 64 | 64 |
| PA 260-97103^{†} | 16 | 2 | 8 | 16 | 2 | 8 | 16 | 8 |
| PA 262-101856^{†} | 32 | 4 | 16 | 2 | 8 | 16 | 4 | 16 |
| PA 264-104354^{†} | 16 | 4 | 8 | 4 | 8 | 16 | 8 | 16 |
| PA 91433^{†,}* | 8 | 2 | 8 | 4 | 8 | 4 | 8 | 8 |
| PA 100036^{†} | 8 | 2 | 8 | 4 | 4 | 8 | 4 | 4 |
| PA 101885^{†} | 4 | 2 | 2 | 4 | 2 | 2 | 4 | 2 |
| PA 101243^{†,}* | 8 | 2 | 8 | 8 | 4 | 8 | 8 | 4 |
| PA 114228^{†,}* | 8 | 2 | 8 | 0.5 | 1 | 8 | 4 | 1 |
| PA 86052^{†} | 32 | 8 | 16 | 16 | 4 | 16 | 16 | 16 |
| PA 88949^{†} | 64 | 8 | 32 | 4 | 64 | 64 | 32 | 64 |
| PA 107092^{†} | 64 | 8 | 16 | 16 | 16 | 32 | 32 | 32 |
| PA 108590^{†} | 16 | 0.25 | 16 | 4 | 0.5 | 16 | 8 | 4 |
| PA 109084^{†} | 16 | 8 | 8 | 8 | 8 | 16 | 16 | 16 |
| PA O200^{#} | 4 | 1 | 2 | 1 | 1 | 2 | 1 | 2 |
| PA O750^{#} | 4 | 0.5 | 2 | 1 | 2 | 4 | 2 | 1 |

^{†}Clinical isolate;
^{‡}MBL-expressing;
*colistin-resistant;
^{#}efflux-deficient.

1. An antibiotic adjuvant compound comprising a structure as set forth in formula (I) 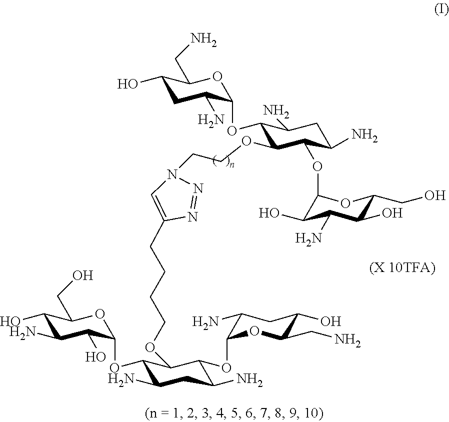
wherein n is an integer between 1 and 10.

2. A method of permeabilizing a Gram Negative bacterium outer membrane comprising:
administering to the Gram Negative bacterium an effective amount of a compound comprising a structure as set forth in formula (I): 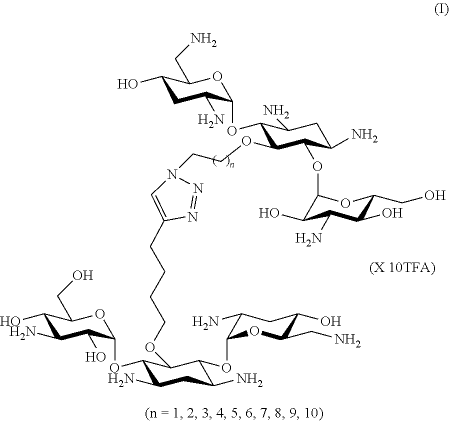
wherein n is an integer between 1 and 10.

3. A method of potentiating antibacterial activity of an antibiotic against a Gram Negative bacterium comprising:
administering an effective amount of a compound of formula (I) 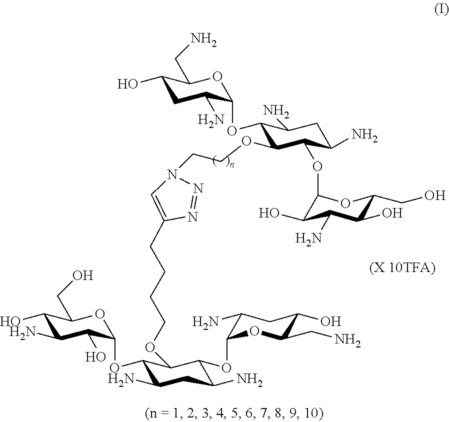
wherein n is an integer between 1 and 10; and an effective amount of an antibiotic selected from the group consisting of: an outer membrane impermeable antibiotic; an efflux-prone antibiotic; a β-lactam antibiotic; fosfomycin or a quorum sensing inhibitor.

4. The method according to claim 3 wherein the OM-impermeable antibiotic is rifampicin, linezolid, clindamycin or novobiocin.

5. The method according to claim 3 wherein the efflux-prone antibiotic is a tetracycline, a fluoroquinolone, or chloramphenicol.

6. The method according to claim 3 wherein the β-lactam antibiotic is a monobactam, a carbapenem, a cephalosporin or a penicillin.

7. The method according to claim 6 wherein the β-lactam is given in combination with a β-lactamase inhibitor.

8. The method according to claim 7 wherein the β-lactamase inhibitor is selected from the group consisting of: vaborbactam, sulbactam, clavulanic, tazobactam, zidebactam, nacubactam, ETX2514, avibactam and relebactam.

9. The method according to claim 3 with the proviso that the antibiotic is not an aminoglycoside or a polymyxin.

10. The method according to claim 3 wherein the quorum sensing inhibitor is a salicylanilide.

11. The method according to claim 10 wherein the salicylanilide is niclosamide, rafoxanide, oxyclozanide or closantel.

12. (canceled)

13. (canceled)

14. (canceled)

15. (canceled)

16. (canceled)

17. (canceled)

18. (canceled)

19. (canceled)

21. (canceled)

22. (canceled)

23. (canceled)
